(19) United States
(12) Patent Application Publication
(10) Pub. No.: US 2026/0348051 A1
(43) Pub. Date: Nov. 5, 2026

(54) COLLAGENOUS FILAMENT

(71) Applicant: FIBRILLOGENESIS LTD, Yeovil Somerset (GB)

(72) Inventors: David O’LEARY, Yeovil Somerset (GB); Darryl CASSINGHAM, Yeovil Somerset (GB)

(21) Appl. No.: 19/484,557

(22) PCT Filed: May 15, 2024
(86) PCT No.: PCT/GB2024/051267

§ 371 Date: Nov. 14, 2025

(30) Foreign Application Priority Data
May 17, 2023 (GB) 2307313.3

(51) Int. Cl. D01D 5/06; D01F 6/68; D02J 13/00

(52) U.S. Cl. CPC D01D 5/06; D01F 6/68; D02J 13/00

(57) ABSTRACT

Disclosed is a method of forming a collagenous textile filament. The method comprises supporting a continuous thread of collagenous extrudate upon a moving build platform surface through the liquid of a fibrillation bath; transferring the thread of collagenous extrudate to at least one drying module; and moving the thread of collagenous extrudate through the or each drying module. In the or each drying module, the thread moves unsupported through a drying cavity from a first roller to a second roller spaced apart from the first roller. The or each drying cavity is warmed by at least one heater. Also disclosed are related apparatus, products and uses of the collagenous textile filament.

### FIELD OF THE DISCLOSURE

The present disclosure concerns collagenous filament. Also disclosed are methods and apparatus for continuously forming a collagenous filament.

### BACKGROUND OF THE DISCLOSURE

There is an increasing awareness of the need to find economic, efficient and effective methods of extracting useful products from waste materials.

In recent years, it has become apparent that not only is there a need to reduce waste, but also that waste may comprise valuable resources. As an example, waste animal matter from tanneries or abattoirs may contain usable materials.

Often, low value animal hides (such as those of cattle which have survived extreme winter conditions, or which have been in contact with barbed wire, and which are therefore badly damaged) are sent to landfill, burnt, buried or otherwise disposed of as being unsuitable for the production of leather.

A material that may be extracted from waste animal matter is collagen. A way to use that collagen is to hydrolyse it, thus forming gelatin which has been used in glues and as a thickener. Another use for collagen is in edible products (such, for example, as collagenous food casings). U.S. Pat. No. 3,433,864 discloses the addition of acid soluble collagen to an aqueous suspension of collagen from limed hide material. The suspension is extruded, coagulated and further processed for use, for example, in food casings.

As a naturally occurring material with little inter-species variation, collagen is also known for its biocompatibility, eliciting low antigenicity and having a low host response risk. As a result it is suitable for medical implementations. U.S. Pat. No. 2,485,958 discloses collagen for use in medical sutures, prepared via extrusion of a solution of collagen through a heated spinneret into a coagulating bath. Collagen has also found application in the manufacture of implantable prostheses, as a cell growth substrate, and in the preparation of living tissue equivalents. Thus, U.S. Pat. No. 5,378,469 discloses extruding a collagen solution into a dehydrating agent to form a medical product. U.S. Pat. No. 5,911,942 discloses the formation of elongate collagenous structures coated with micro-particulate animal tissue, also for use in bioengineering. Zeugolis et al., J. Biomat. Sci., 2009, 20, 219-234 discloses threadlike collagen products with properties suitable for tissue engineering applications and the effect of salt concentration during the production of the products is reported. The same authors have investigated the effect of different collagen sources (bovine or rat tendon) as well as different extraction methods (acid or peptin solubilisation of the tendon) on the quality of the threadlike collagen product and its suitability for biomedical use (Zeugolis et al., J. Biomed. Mat. Res., 2007, 86A, 4, 892-904). More recently, Haynl et al., Nano. Lett., 2016, 16, 5917-5922 investigated threadlike collagen products produced using a microfluidic device instead of the more conventional wet-spinning methods described, for example, in US 2018/0193524 A1. Picaut et al., Biomed Phys. & Eng. Express, 2018, 4 (3), pp. 035008 likewise concerns threadlike collagen products for use in tissue engineering applications. In pursuit of a synthetic tendon, Picaut et al. extrude collagen solutions into buffers. Tonndorf et al., Biomed. Mat., 2019, 14, 039501 similarly concerns reconstituted collagen products for biomedical applications; riboflavin-induced photo-crosslinking of collagen is explored as an alternative to glutaraldehyde crosslinking for the preparation of wet spun threadlike collagen products.

However, waste-derived collagen may be limited as to its biomedical application. Waste-derived collagen has, therefore, typically been used for glue or to produce gelatin as described above. Some attempts have been made to repurpose waste animal hides, or even leather (processed from hide) in other ways. For example, U.S. Pat. No. 8,328,878 describes loosing collagen from leather and mixing it with cotton fibres; the resultant blend is spun into a staple yarn. Similarly, EP 4092171 A1 discloses grinding leather then siphoning fine fibres from the ground material and blending them with polyester to form a staple yarn. Meanwhile, US 2020/0048794 A1 discloses spraying a collagenous solution onto the surface of a belt or cylinder, where it forms a reconstituted collagenous network, which is harvested from the surface, forming staple fibres for a wool-like material. U.S. Pat. No. 9,109,326 describes collagen powder obtained by passing collagenous stock solution through a spinning nozzle into a series of baths then drying bundles of the resultant threadlike collagen product in a convection oven at 50 °C before pulverising them.

At the time of writing, valuable collagen present in waste is still not fully utilised and there remains a need to utilise such collagen in a more effective way. Meanwhile, there is a need to provide more effective ways for processing solutions of collagen in water into useful products.

### DESCRIPTION OF THE DRAWINGS

In the accompanying drawings:

FIG. 1 shows a schematic cross-section of animal hide.

FIG. 2A shows a side view of an extrusion pressure vessel.

FIG. 2B shows a perspective view of the extrusion pressure vessel of FIG. 2A.

FIG. 3A shows a perspective view of a fibrillation bath (vessel and liquid) containing a moveable build platform.

FIG. 3B shows a side cross-sectional view of the fibrillation bath of FIG. 3A.

FIG. 3C shows a top view of the moveable build platform of FIG. 3A.

FIG. 3D shows a perspective view of the bath of FIG. 3A without the moveable build platform.

FIG. 3E shows a perspective view of an alternative moveable build platform.

FIG. 3F shows a side view of the moveable build platform of FIG. 3E.

FIG. 3G shows a perspective enlarged view of guide channels to guide threads extruded onto the surface of the moveable build platform of FIGS. 3E and 3F.

FIG. 3H shows a perspective view of a fibrillation bath (vessel and liquid) containing the moveable build platform of FIGS. 3E to 3G.

FIG. 4A shows a side view of a drying module.

FIG. 4B shows a simplified side view of the drying module of FIG. 4A.

FIG. 4C shows a perspective view of the drying module of FIG. 4A.

FIG. 4D shows a simplified perspective view of the drying module of FIG. 4A.

FIG. 4E shows a further-simplified side view of the drying module of FIG. 4A.

FIG. 4F shows a top view of a heater of the drying module of FIG. 4A.

FIG. 4G shows a simplified side view of a plurality of drying modules of FIG. 4A arranged in series.

FIG. 4H shows a simplified perspective view of plurality of drying modules of FIG. 4A arranged in series.

FIG. 4I shows a further-simplified side view of a plurality of drying modules of FIG. 4A arranged in series.

FIG. 5A shows a side view of a winder.

FIG. 5B shows a perspective view of the winder of FIG. 5A.

FIGS. 6A and 6B show schematic views of apparatuses for forming collagenous textile filaments.

FIG. 7A is a photograph of a multifilament yarn consisting of collagenous filaments in accordance with the fourth aspect of the present disclosure.

FIG. 7B is a photograph of the multifilament yarn loaded onto a loom in preparation for weaving.

FIG. 7C is a photograph of a textile being woven on the loom from the multifilament yarn.

FIG. 7D is a magnified section of FIG. 7C.

FIG. 7E is a photograph of the woven textile once finished and removed from the loom.

### SUMMARY OF THE DISCLOSURE

“Collagen”, as the term is used herein, refers to three polypeptide chains aligned in an elongate triple helix (also referred to in the art as tropocollagen), which may have a length in a range of from about 10 to about 500 nm (such as about 300 nm) and may have a diameter in a range of from about 0.5 to about 5 nm (such as about 1.5 nm). The association between polypeptide chains within and between tropocollagens may be due to hydrogen bonding, van der Waals forces, ionic bonding and/or inter-chain covalent bonding, such as bonding which results from condensation reactions between lysine and hydroxylysine residues. Cross-linking between lysine and/or hydroxylysine residues may result from the oxidative deamination of the ε-amino groups of said residues, for example in the presence of lysyl oxidase or in the presence of a cross-linking agent, such as glutaraldehyde.

Collagen in accordance with the present disclosure may be or comprise one or more fibrous collagens. Collagen in accordance with the present disclosure may be or comprise Type I collagen. Type I collagen may form a major proportion by weight of the collagen, such as at least about 70% w/w, preferably at least about 80% w/w, of the collagen of the present disclosure.

A collagen of the solution of collagen in water as disclosed herein may be or comprise acid-solubilised collagen (ASC). Collagen encompasses ASC. ASC is known in the art, see for example Jongjareonrak et al., Food Chemistry, 2005, 93(3): 475-484, incorporated by reference herein. ASC may comprise or consist essentially of intact tropocollagen molecules, optionally with different amino acid residues' charges, and/or different association between polypeptide chains within and between tropocollagens, compared to non-acid-solubilised collagen.

As used herein, the term collagen fibril refers to an elongate structure formed of a plurality of (tropo-) collagen molecules bound together; optionally having a diameter in a range of from about 10 to about 100 nm. In the biochemical arts, the term “collagen filament” may sometimes be used to describe sub-structures of collagen fibrils and “collagen fibre” may sometimes be used to describe super-structures of collagen fibrils. It will, however, be understood that in the present specification, the terms “fibre” and “filament” have their normal meaning in the textile arts, for example as defined in Understanding Textiles, 7^{th} Ed., Collier et al., 2009 and in Textile Science, Hatch, 1993, both incorporated herein by reference in their entireties. Thus, a fibre as the term is used herein is a textile fibre, meaning an elongate strand formed of polymer molecules and which is configured for use in textiles. Apart from polymer molecules themselves, a fibre may, in the textile arts, be the smallest unit typically referred to. A filament as the term is used herein is a textile filament, being configured for use in textiles. Textiles include any woven, non-woven and knit textiles. Leather is not a textile, although leather-like textiles are known that are synthetic materials that are used as substitutes for leather, as described in Textile Science, Hatch, 1993.

As used herein, “staple” refers to short lengths of fibre (especially, having a length in a range of from about 1 mm to about 200 mm) which have an intrinsic limit to their length; for example, because of the process by which they have been formed, or because when grown in nature they can only ever attain up to a certain length. Examples include wool, fur fibre and cashmere.

As used herein, “filament” refers to a continuous fibre; “continuous” is a descriptor commonly used in the textile arts, and means the fibre has no intrinsic limit to its length, although it may be cut to a particular length if desired. Typically a filament may have a length of at least about 500 mm. Examples include polyester, spider silk and olefinic filaments. The collagenous filament of the present disclosure falls into this category.

The present disclosure provides, according to a first aspect, a method of forming a collagenous textile filament, comprising: supporting a continuous thread of collagenous extrudate upon a moving build platform surface through the liquid of a fibrillation bath; transferring the thread of collagenous extrudate to at least one drying module; and moving the thread of collagenous extrudate through the or each drying module, wherein in the or each drying module the thread moves unsupported through a drying cavity from a first roller to a second roller spaced apart from the first roller, wherein the or each drying cavity is warmed by at least one heater.

In a second aspect, the present disclosure provides apparatus for forming a collagenous textile filament, comprising: a fibrillation bath vessel containing a moveable build platform; and at least one drying module downstream of the fibrillation bath vessel, the moveable build platform having a surface configured to receive a continuous thread of collagenous extrudate and to move thereby supporting the received continuous thread of collagenous extrudate through the liquid of a fibrillation bath; the or each drying module comprising a first roller and a second roller spaced apart from the first roller, a drying cavity between the rollers through which the continuous thread of extrudate is moveable unsupported during operation, and at least one heater configured to warm the or each drying cavity.

In a third aspect, there is provided a method of forming a collagenous textile filament, comprising operating apparatus defined in accordance with the second aspect of the disclosure.

Preferably, the at least one drying module is a plurality of drying modules, especially in a range of from about 5 to about 3000 drying modules.

“Upstream” and “downstream” are used herein relative to the motion of the continuous thread of collagenous extrudate. The thread moves, when the apparatus is in use, from upstream to downstream. Thus, for example, thread moves from the build platform surface to the downstream drying module(s).

“Horizontal” and “vertical” are used herein relative to the fibrillation bath liquid. Thus, “horizontal” means substantially parallel to the surface of the liquid. “Vertical” means substantially perpendicular to the surface of the liquid.

The term “bath” is used herein to refer to a vessel containing a liquid. A fibrillation bath vessel is a vessel for such a bath. A fibrillation bath liquid is a liquid for such a bath. A spraying device for spraying fine droplets of liquid at a thread of collagenous extrudate or at a collagenous textile filament, does not equate to a bath.

The extrudate according to the present disclosure may contain collagen in a range of from about 0.1% w/w to about 5% w/w, preferably of from about 0.5% w/w to about 3% w/w, most preferably in a range of from about 0.5% w/w to about 1.5% w/w. It has been found that the higher the % w/w collagen present, the more viscous the extrudate. If the collagenous extrudate has a concentration of collagen that is too high, for example of above about 5% w/w, the pressure required to form it by extrusion may, for some types of apparatus, be impracticable. If the collagenous extrudate has a concentration of collagen that is too low, for example of below about 0.1% w/w, it may be impracticable (such, for example, as too delicate or too costly) to dry.

Immediately after it has been extruded, the disclosed thread of collagenous extrudate may be prone to breakage, being weak and unable to support its own weight, whether in air or in higher density environments such as when immersed in the liquid of a bath. This weakness is thought to be due to the high water content of the collagenous extrudate and corresponding low levels of bonding between collagen molecules (prior to fibrillation and cross-linking). The collagenous extrudate may have a water content in a range of above about 80% w/w, especially above about 90% w/w, more especially above about 95% w/w.

It has now been found that when a thread of collagenous extrudate (from an extrusion needle) is received upon the surface of a moveable build platform which then supports the thread of collagenous extrudate through the liquid of a fibrillation bath, the surface bears up the thread of collagenous extrudate and protects its integrity while it is fibrillating within the liquid of the bath. The build platform may, for example, support the thread for a period of at least about 30 minutes, especially at least about 1 hour; optionally up to a maximum of about 3 hours, especially a maximum of about 2 hours, within the liquid of the bath. The residence time in the liquid of the bath is preferably in a range of from about 1 hour to about 2 hours. The supportive surface may prevent or mitigate breakage of the continuous thread of collagenous extrudate, enabling the thread to remain continuous while fibrillation develops and it gains integrity and strength. In contrast, collagenous extrudate unsupported in a liquid of a fibrillation bath is liable to break. By supporting the thread on a surface, improved conditions for fibrillation are provided so that the thread may be transferred from the liquid of the fibrillation bath to one or more drying modules after no more than about 2 hours, especially no more than about 1.2 hours. Improved crystallinity of the collagenous structure of the thread may be provided due to the increased opportunity for collagen molecules to align in the thread. This may provide improved strength of the resultant collagenous textile filament.

As used herein, the term fibrillation refers to the assembly (especially, the auto-assembly) of collagen molecules into higher order structures, especially fibrils as defined herein. Thus, fibrillation may impart strength and integrity.

Meanwhile, tanning refers to cross-linking of collagen molecules, especially cross-linking of lysine residues therein, for example using glutaraldehyde; also imparting strength. Thus, the collagenous filament disclosed herein is preferably a tanned collagenous filament.

When the thread is passed to the drying module, having undergone fibrillation, it may be strong enough to be gently handled, e.g. with tweezers. However, it is not yet in the form of a collagenous filament that is dry to the touch. Instead, it remains weak and unable fully to support its own weight.

Preferably, the liquid of the fibrillation bath is an aqueous tanning and fibrillation solution. Simultaneous exposure of the collagen thread to both fibrillation agents and tanning agents in an aqueous solution may provide a gentle way to strengthen the collagenous structure therein. Aqueous solutions, as opposed to solutions based on other solvents such as ethanol or acetone, may avoid damage to the collagen. Simultaneously exposing collagen to both fibrillation agents and tanning agents at the same time may obviate the need for multiple baths in series. Thus, preferably, the thread of collagenous extrudate is exposed to substantially no organic solvents (more preferably, it is not exposed to organic solvents) before it has been made into a (dry) collagenous filament by the method of the invention, and the apparatus of the disclosure is configured accordingly.

Although, preferably, the liquid of the fibrillation bath is an aqueous tanning and fibrillation solution so that the thread has absorbed tanning (cross linking) agents from the solution, the low concentration of collagen (due to the high concentration of water) still in the thread, may mean that only a little cross linking may have been able to occur, since collagen molecules may have remained far apart. Upon drying in the drying module, the collagen can be cross linked, water can evaporate and a strong collagenous filament can form, which is dry to the touch and able to support its own weight across several hundred metres or more.

Advantageously, the continuous thread of extrudate is moveable unsupported through the drying cavity or cavities. As it is not in contact with surfaces while it is being dried, it is not moulded (for example, into a flat, ribbon-like shape) during drying, by surfaces. Instead, it retains the cross-sectional shape imparted to the thread of extrudate upon extrusion, such as a substantially round cross-sectional shape, as may be desirable for filament configured for use in textiles. This may enable the cross-sectional shape of the thread to be determined by selecting an appropriate extrusion needle cross-sectional shape. It has also been found that supporting the thread on the build platform surface does not adversely impact the thread cross-sectional shape (for example, because the thread is very slightly buoyed by the fibrillation bath liquid and/or because the thread is less liable to change shape in the bath when it has not lost much, if any, water content, compared to when it is drying and losing water content thus changing diameter). Similarly, when thread is supported in contact with other thread in the way disclosed hereinbelow, it has been found that this does not adversely impact the thread cross-sectional shape.

Therefore, the moveable build platform and drying module(s) work together to produce a strong collagenous filament, able to support its own weight, from collagenous extrudate unable to support its own weight, and having a desirable cross-section. Thousands of metres (e.g., at least about 10,000 metres) of continuous collagenous filament may be produced in this manner.

The drying module(s) obviate the need for drying baths, such as vessels filled with organic solvents, which can damage collagen. Thus, preferably, there are no organic solvent baths upstream of the drying module(s).

Without wishing to be bound by theory, it is thought that not only cross-linking of collagen, but also extensive hydrogen bonding, may contribute to the strength of the continuous collagenous filament. By using a fibrillation bath immediately (i.e., directly) upstream of the drying module(s) (thus, with no intermediate drying baths, especially those with organic bath liquid, such as ethanol or acetone based baths) the apparatus and method of the disclosure are configured to encourage hydrogen bonding of the collagen.

It has been found that, subsequent to the drying module(s), the collagenous filament of the disclosure may be resistant to contact with moisture, so that the network of hydrogen bonding does not disintegrate if the collagenous filament is re-contacted with water. The collagenous filament may have a negligible or low moisture regain relative, for example, to natural leather. The collagenous filament may be resistant to damage by microorganisms.

The method according to the first aspect of the present disclosure may comprise a step of extruding the continuous thread of collagenous extrudate from an extrusion needle (also referred to as an extrusion nozzle) into the liquid of the bath and towards (especially, onto) the surface of the moveable build platform, before supporting the thread upon the moving build platform surface through the liquid of the bath. Relatedly, the apparatus according to the second aspect of the present disclosure may comprise an extrusion needle for extruding the continuous thread of collagenous extrudate into a liquid of a fibrillation bath and towards (especially, onto) the surface of the moveable build platform. The extrusion needle, or at least a tip thereof, may be submerged (in the method)/ submergible (in the apparatus) in the liquid of the fibrillation bath (preferably, for the entirety of the needle's operation).

In the method and apparatus according to the present disclosure, the extrusion needle may be moved or moveable relative to the surface of the moveable build platform; such as in a plane parallel to the moveable build platform and/or in a plane perpendicular to the moveable build platform, especially in a plane parallel to the moveable build platform. Most preferably, the needle moves or is configured to move in a plane parallel to the surface of the moveable build platform; and the build platform surface moves or is configured to move horizontally or vertically through the liquid of the fibrillation bath.

When the rate of extrusion of the thread of collagenous extrudate into the liquid of the bath (through the extrusion needle) is at a speed in a range of 0.5 to 200 m/min, the speed of movement of the needle relative to the surface of the moveable build platform may be in a range of from 0.1 mm/s to 100 mm/s, e.g. for 1 m/min extrusion the needle may move relative to the surface at 20 mm/s. This may help avoid breakage of the extrudate between exiting the needle and contacting the build platform surface.

Optionally, both the movement of the needle relative to the build platform surface and the speed of extrusion of the thread of collagenous extrudate into the liquid of the bath are synchronised (in the method) or configured to be synchronised (in the apparatus) with the movement of the surface of the build platform. This may promote the continuous formation of a collagenous textile filament without it breaking or being unduly strained.

Where reference is made to synchronisation, that may mean movement at the same or substantially the same speeds. However, the term encompasses different speeds which are configured together to provide the correct tensioning of the thread of collagenous extrudate to prevent or mitigate stretching or breaking it. Synchronisation may be achieved by motor systems connected to a control system.

The extrusion needle may vary in the distance at which its tip is situated from the surface of the moveable build platform. For example, the distance may vary when the needle is moved or moveable in a plane parallel to the surface of the moveable build platform and the build platform is moved or moveable vertically.

Preferably, there may be a plurality of extrusion needles, arranged together in the disclosed method and apparatus to extrude a corresponding plurality of threads of collagenous extrudate towards (especially, onto) the build platform surface. Each thread in the plurality of threads may be transferred from the build platform surface to a respective drying module (i.e., there is at least one drying module and optionally several drying modules, for receiving each thread; a drying module does not receive more than one thread), for example using a transfer rod capable of picking up and moving the entire plurality of threads at once (each to their separate, respective drying modules). Optionally, each thread in the plurality moves through more than one drying module, especially more than about 5 drying modules, preferably in a range of from about 5 to about 3000 drying modules. The plurality of more than one drying modules may be arranged in a grid. For example, if there are 20 extrusion needles extruding 20 threads, there may be provided a 20 by 1600 grid of drying modules.

Thus, the method may preferably comprise extruding a plurality of continuous threads of collagenous extrudate from a corresponding plurality of extrusion needles into the liquid of a fibrillation bath and towards (especially, onto) the surface of a moveable build platform positioned within a fibrillation bath vessel (i.e., all of the plurality of threads are supported on the same surface of the same moveable build platform); supporting the plurality of threads upon the moving build platform surface through the liquid of the bath; and then carrying out the following steps in respect of each thread of collagenous extrudate: transferring the thread of collagenous extrudate to at least one drying module; and moving the thread of collagenous extrudate through the or each drying module, wherein in the or each drying module the thread moves unsupported through a drying cavity from a first roller to a second roller spaced apart from the first roller, wherein the or each drying cavity is warmed by at least one heater.

Similarly, the apparatus may preferably comprise: a plurality of extrusion needles for extruding a plurality of continuous threads of collagenous extrudate into a fibrillation bath liquid and towards (especially, onto) the surface of a moveable build platform in a fibrillation bath vessel; said fibrillation vessel containing said moveable build platform, said surface being configured to receive the plurality of continuous threads of collagenous extrudate and to move thereby passing the received plurality of continuous threads of collagenous extrudate through the liquid of the fibrillation bath; and, in respect of each thread of collagenous extrudate for which the apparatus is configured, at least one drying module downstream of the moveable build platform, the or each drying module comprising a first roller and a second roller spaced apart from the first roller, a drying cavity between the rollers through which the continuous thread of extrudate is moveable unsupported during operation, and at least one heater configured to warm the or each drying cavity.

Preferably, the at least one drying module in respect of each thread of collagenous extrudate is a plurality of drying modules, especially in a range of from about 5 to about 3000 drying modules.

Preferably, when there is a plurality of continuous threads of collagenous extrudate, the drying modules are arranged in a grid of X by Y drying modules, wherein X represents the number of collagenous threads, and Y represents the number of drying modules for each collagenous thread.

Whether in the case of a single thread or a plurality of threads, the extrusion of any given thread towards (especially, onto) the surface may be guided by a guide channel. Thus, the method may comprise guiding the or each thread from the or each needle towards the build platform surface via a guide channel. The or each guide channel may be coupled with the fibrillation bath vessel. A guide channel may help position thread more precisely thus mitigate breakage, undue strain or misshapenness; when there are a plurality of threads, guide channels may help prevent contact between threads extruded by adjacent extrusion needles.

The build platform may especially have a substantially horizontal surface configured to slot into place amongst a plurality of guide channels configured to guide a corresponding plurality of extruded threads, the guide channels being coupled to the fibrillation bath vessel.

The surface of the build platform, especially when there are a plurality of threads (and, preferably, guide channel(s) for the or each thread) may be substantially polygonal or substantially circular. If so, preferably the (or each, when there are a plurality of threads) extrusion needle is configured to extrude thread towards (especially, onto) a substantially polygonal or substantially circular path the surface.

The apparatus may be configured for a first length of thread to be supported directly on the build platform surface and for a second length of the same, continuous thread to be supported on the first length of thread on the build platform surface. Similarly, in the method, a first length of thread may be supported directly on the build platform surface and a second length of the same, continuous thread may be supported on the first length of thread on the build platform surface. The time interval between extruding the first length and extruding the second length may preferably be in a range of from about 1 second to about 1 minute, for example about 3 seconds. By the time the second length has sunk through the fibrillation bath liquid into contact with the first length, the external surface of the first length may be fibrillated just enough for its cross-sectional shape to be substantially unaffected by supporting the second length of thread.

The extrusion needle(s) may have an inner diameter in a range of from about 0.1 to about 5 mm, especially 0.5 mm to about 2 mm, for example about 1 mm.

The collagenous extrudate may be or comprise a gel of collagen in water, having a collagen content in a range of from about 0.1 to about 5% w/w, especially about 0.5 to about 3% w/w, for example about 1.25% w/w. Thus, the needle may be configured to extrude such a gel.

Optionally, the collagenous extrudate is extruded under an extrusive pressure in a range of from about 2 to about 10 bar, especially about 3 to about 7 bar, for example about 6 bar. Thus, the needle(s) may be configured to operate under such an extrusive pressure.

Optionally, during extrusion, the collagenous extrudate is at a temperature in a range of from about 0 to about 10 °C, especially about 1 to about 5 °C, for example about 3 to about 4 °C. Thus, the needle(s) may be configured to operate at such a temperature. Such temperatures may help further stabilise the extrudate. Optionally, the fibrillation bath liquid is also at such a temperature.

The extrusion of the thread may be at a speed in a range of from about 0.1 m/min to about 200 m/min, especially about 0.5 m/min to about 150 m/min. Speeds in this range may mitigate the risk of breakage, ensuring the formation of a single continuous collagenous filament from the collagenous extrudate. The apparatus may be configured accordingly. As described hereinabove, the movement of the build platform may be synchronised with the speed of extrusion and the movement of the extrusion needle(s).

The extrusion needle may be connected via airtight tubing to an extrusion vessel comprising a solution of collagen in water for forming the collagenous extrudate (when there is a plurality of extrusion needles, each extrusion needle may be connected via airtight tubing to a respective extrusion vessel comprising a solution of collagen in water for forming the collagenous extrudate). Preventing the entry of air bubbles into the tubing may ensure a continuous thread of extrudate is formed; air bubbles may risk disrupting the extrudate and may risk the formation of a discontinuous thread. Optionally, the tubing has a low-friction internal surface (i.e., a surface having a coefficient of static friction with the solution of collagen in water in a range of from about 0.01 to about 0.10), such as a PTFE surface, thereby enabling solution to flow freely through it. Optionally, the tubing comprises one or more bubble traps. The tubing may comprise one or more microporous membrane (especially, microporous PTFE membrane) bubble traps, especially those being operable under negative pressure (such as a negative pressure in a range of about 1 to about 1000 mbar). The solution, if it contains bubbles, may be fed into the one or more traps; bubbles may then be sucked through the microporous membrane, advantageously without leaking of the solution; optionally, when a negative pressure is to be used, a vacuum pump may be connected to an outlet of the trap.

The extrusion vessel may comprise a container having a substantially conical or substantially frustoconical portion, which is connected to the tubing and configured to hold the solution of collagen in water, optionally at a temperature in a range of from about 0 to about 10 °C, especially about 1 to about 5 °C, for example about 3 to about 4 °C. The extrusion vessel may comprise a temperature control device; especially, at least the substantially conical or substantially frustoconical portion may be housed in a temperature control chamber. The temperature may help to prevent degradation of the collagen. Additionally or alternatively, it may help to reduce the solubility of any remaining air present within the solution of collagen in water, to mitigate or avoid the formation of discrete air bubbles. Keeping the temperature constant or within a small range of temperatures may help to ensure that the viscosity of the solution remains constant and therefore its speed through the tubing remains constant.

The substantially conical or substantially frustoconical portion, when holding the solution of collagen in water, may help to prevent or mitigate cavitation of the solution during extrusion, thus may prevent the formation of air bubbles therein, decreasing the risk of discontinuities in the thread.

Optionally, in the method, the solution of collagen in water may be centrifuged at about 1500 to about 3500 rpm, especially about 2000 to about 3000 rpm. Centrifugation may occur prior to loading the solution of collagen in water into the extrusion vessel. Thus, optionally, the apparatus comprises a centrifuge (preferably configured for centrifugation at about 300 to about 4000 rpm) upstream of the extrusion vessel.

Optionally, in the method or apparatus, the solution of collagen in water in the extrusion vessel is under positive pressure. This may be useful for driving extrusion. Optionally, the positive pressure is in a range of from about 3000 to about 10,000 mbar, especially about 3000 to about 7000 mbar, more especially about 4000 to about 6000 mbar, for example about 5,450 mbar. Optionally, the pressure is calculated based on the desired speed of extrusion.

The build platform surface may be configured to hold the thread in place by friction. The build platform surface may have a coefficient of static friction with the thread in a range of from 0.5 to about 0.95, especially about 0.7 to about 0.9. Optionally, the build platform surface is abrasive or otherwise textured. The build platform surface may be flat or substantially flat. The build platform surface may be a surface of a conveyor belt, but it is not a surface of a cylinder, such as a roller. It will be understood that the build platform surface has an area sufficient to fulfil its function of supporting the thread. Thus, the build platform surface for supporting the thread may have a surface area of at least about 0.4 m², preferably at least about 5 m².

It is most preferred for the build platform surface to be configured to be horizontal (relative to the fibrillation bath liquid). Otherwise, it may be difficult for the surface to support the thread(s) properly, under the influence of gravity.

Supporting the thread upon the moving build platform surface through the liquid of the fibrillation bath may comprise moving the surface through the liquid horizontally.

Supporting the thread upon the build platform surface through the liquid of the bath may comprise moving the surface through the liquid of the bath vertically.

As disclosed herein, the build platform surface may be a surface of a conveyor belt. The build platform surface may be or comprise a flexible surface, for example a surface formed from a material having a Young's modulus in a range of about 0.1 GPa or below. For example, the build platform may be a conveyor belt configured to flex. When the build platform surface is a surface of a conveyor belt, it may, especially, move or be moveable horizontally.

The build platform surface may be or comprise a rigid surface, for example being formed from a material having a Young's modulus in a range of about 10 GPa or above. When the build platform surface is a rigid surface, it may, especially, move or be moveable vertically. Especially when the build platform surface is a rigid surface, the fibrillation bath vessel may be coupled to the or each guide channel described hereinabove.

The build platform surface is preferably configured to be substantially horizontal during use. The substantially horizontal surface may be configured to move horizontally or vertically. Having a horizontal surface enables the thread to be supported, while having a moveable surface enables the thread to be transported and effectively supported. Thus, the thread may be transported through the liquid of the bath, fibrillating as it moves, without breaking or stretching; then be delivered to the drying module(s).

The build platform surface may be a perforated surface, although it will be understood this does not interfere with its function of supporting the thread. Liquid of the bath may be able to pass through the perforations as the surface moves, thereby reducing the resistance to movement of the surface, especially if the surface is a horizontal surface that moves or is moveable vertically, as described herein. Thus, the surface may be a perforated horizontal surface that moves or is moveable vertically. The perforations may be microscopic or macroscopic but preferably they are macroscopic. The perforations may advantageously be configured to not interfere with the supporting action of the surface for the thread. It will be understood that thread does not pass through the perforations if present.

In the method according to the first aspect of the present disclosure, the liquid of the fibrillation bath may, most preferably, be an aqueous fibrillation and tanning solution. Accordingly, the liquid of the bath may comprise both one or more fibrillation agents and one or more cross-linking agents. Similarly, the apparatus may further comprise the liquid of the fibrillation bath, especially an aqueous fibrillation and tanning solution, which comprises both one or more fibrillation agents and one or more cross-linking agents, contained within a fibrillation bath vessel.

The one or more fibrillation agents may comprise one or more buffer systems, especially a sodium tetraborate/boric acid buffer system (e.g., at a pH in a range of about 7.5 to about 8.5, especially of about 8.0). Other such buffer systems include, without limitation, phosphate buffer systems (e.g. at a pH in a range of about 5.5 to about 8.5, especially about 5.8 to about 8.0); EDTA buffer systems (e.g., at a pH in a range of about 7.5 to about 8.5, especially of about 8.0) and potassium dihydrogen phosphate/sodium hydroxide buffer systems (e.g., at a pH in a range of about 7.5 to about 8.5, especially of about 8.0).

Additionally or alternatively, the one or more cross-linking agents may comprise one or more cross-linkers of lysine residues, since collagen comprises a plurality of such residues. A preferred example is gluteraldehyde. Other examples include ortho-phthalaldehyde (OPA) and polycarbamoyl sulfonate (PCMS).

The build platform surface may be submerged at a level of at least about 5 mm, especially at least about 10 mm, below the surface of the liquid of the bath.

The build platform is not simply the bottom of the fibrillation bath vessel.

Suitably, the fibrillation bath may be the only bath to which the thread is exposed between extrusion and transfer to the drying module(s). Suitably, the fibrillation bath may be the only bath used in the method and present in the apparatus. This may especially be the case when the liquid of the fibrillation bath is an aqueous fibrillation and tanning solution.

Preferably, the liquid of the fibrillation bath, especially when the liquid is an aqueous fibrillation and tanning solution, is at a pH in a range of from about 7.5 to about 9, especially about 7.6 to about 8.4, for example about 8.

Optionally, transferring the continuous thread of collagenous extrudate from the bath to a (first) drying module comprises transferring the thread via one or more (for example, about 2 to about 4, e.g. about 4) take-off rollers to the drying module. Optionally, the take-off rollers rotate at a speed synchronised with the movement of the build platform surface and the movement through the drying module(s).

Optionally, when there is a plurality of threads as described hereinabove, transferring them to their respective drying modules comprises transferring them via a take-off rod to their respective drying modules.

Optionally, at the point of being transferred to the drying module(s), the thread of collagenous extrudate has an elongation at break in a range of from about 25% to about 40%. Optionally, at that point, the thread of collagenous extrudate still has a % w/w of water in a range of about 90% or above, especially about 95% w/w or above, more especially in a range of about 98.5% w/w or above. The apparatus may be configured to support the transfer of such thread to the drying module(s).

Adequate strength of a collagenous filament for its subsequent use in textiles may, according to the present disclosure, be achieved only subsequent to drying in the drying module(s). Accordingly, gentle handling of the thread of collagenous extrudate during drying may usefully avoid breakage. However, drying in contact with supportive surfaces (even a shaped surface or a low surface energy surface) may have the disadvantage that the filament takes the shape of the surface, drying into a filament that is a flat ribbon instead of having a cross-sectional shape corresponding to that with which it was extruded, such, for example, as a substantially round cross-sectional shape.

All drying module rollers present in the method and apparatus are, preferably, configured to rotate at speeds which are synchronised with each other. As a result, the tension of the thread may be kept constant throughout all drying modules, mitigating or eliminating the risk of breaking or excessively stretching the thread at that stage. However, being synchronised may not necessarily mean they rotate at the same speed. Thus, optionally, the second roller of any given drying module rotates or is configured to rotate at a speed greater than the speed of the first roller of the same drying module to move the continuous thread of collagenous extrudate in the drying cavity. The increase in speed from the first to the second roller may provide improved tensioning of the thread; and, optionally, may facilitate draw or plastic stretching of the filament.

There may be more than one drying cavity in a drying module (preferably, there are at least about two, such as about two, drying cavities in each drying module).

Additionally or alternatively, there may be more than one drying module per thread. Thus, a given thread (whether there is only one thread or a plurality of threads as described herein) may pass over more than two rollers. The first drying module roller contacted by the thread after it exits the liquid of the bath may be, or be configured to be, the slowest drying module roller it contacts; the last drying module roller contacted by the thread before it leaves the drying module(s), e.g. to be wound on a winder, may be, or be configured to be, the fastest drying module roller it contacts. Intermediate drying module rollers may increase in speed from one to the next. This may help to keep tension in the thread as it dries, helping to ensure uniformity along the length of the thread/prevent some parts of the thread stretching more than others. The arrangement may be under feedback control, as an operator may be able to adjust the speeds of the drying module rollers.

Preferably, each drying module roller is configured to rotate at a greater speed than the drying module roller immediately upstream of it, and at a lesser speed than the drying module roller immediately downstream of it. Any given thread may pass through drying module rollers of increasing speeds.

An advantage of a modular drying system is that the number of drying modules can be varied to control the properties of the collagenous textile filament that results. The more drying modules there are, the more quickly the thread of collagenous extrudate can be moved through them.

A drying cavity is configured to dry the thread unsupported, thus advantageously enabling it to dry with substantially the same cross-sectional shape as that of the thread upon extrusion, especially a substantially round cross-sectional shape. However, the drying cavity may also be configured so that the thread is not unsupported for so long a period that it risks breaking or otherwise deforming. Thus, the drying cavity may be elongate and have a length, along which the thread travels unsupported during operation, in a range of from about 100 mm to about 1000 mm, especially about 100 mm to about 500 mm, for example about 200 mm. It has been found that elongate drying cavities with a length in this range may enable drying thread while maintaining substantially the same cross-sectional shape as that of the thread upon extrusion while, at the same time, preventing or mitigating the risk of thread breakage.

The at least one heater may comprise a plurality of air channels encircling or part-encircling the drying cavity. Optionally, each air channel opposes another air channel situated across the drying cavity from it. Air may be heated and fed through the air channels at a low speed, especially a speed in a range of about 0.1 to about 2 m/s, more especially about 0.4 to about 1 m/s, for example about 0.8 m/s. The provision of equal and opposing air channels, encircling or part-encircling the drying cavity (and thus, in use, the thread) may enable hot air within the drying cavity to have a net velocity of about 0 m/s (especially, air at the surface of the thread may have a net velocity of about 0 m/s). Thus, the net air velocity within the drying cavity itself may be in a range of from about 0 m/s to about 1 m/s. This may reduce vibrational and/or sideways motion of thread as it dries. Preferably, therefore, the thread substantially does not ripple as it dries.

Optionally, the heaters warm the drying cavity (in the method) or are configured to warm the drying cavity (in the apparatus) up to a temperature in a range of from about 50 to about 70 °C, especially about 50 to about 67 °C; but no higher. This may help to dry the thread without denaturing the collagen therein.

Optionally, the at least one heater comprises an air diffuser. Air diffusers may help to make the air flow more gentle, thereby reducing vibrational and/or sideways motion of thread as it dries.

In a fourth aspect, there is provided a collagenous filament produced by the method of the first or third aspect of the disclosure.

The collagenous filament is a continuous fibre (in contrast to a staple fibre) comprising (more preferably, consisting essentially of) collagen. It may preferably be a monofilament. It may preferably be dry to the touch. Being collagenous means collagen makes up at least about 70% w/w, preferably at least about 80% w/w, more preferably at least about 90% w/w of the collagenous filament.

Optionally, although being dry to the touch, the collagenous filament comprises water as a minor constituent, such as at most about 30% w/w, preferably at most about 20% w/w, more preferably at most about 10% w/w. Thus, it is especially preferred for the collagenous filament to comprise at least about 90% w/w collagen and at most about 10% w/w water. It will be appreciated that further processing, for example dyeing, fatliquoring, waterproofing and the like, may alter the water content of the filament.

It will be appreciated that the collagenous filament may comprise one or more trace impurities, which may help to identify it, e.g. to identify that it was made by the method of the first or third aspect of the present disclosure, and/or to identify its source, e.g. that it originates from lime split. The term “lime split” as used herein and further described hereinbelow, has its normal meaning in the art, for example in line with ISO 15115:2019, incorporated herein by reference.

The source of the collagenous filament, especially the species of animal from which it derives, may be identified using peptide mass fingerprinting. (An example of peptide mass fingerprinting is zooarchaeology by mass spectrometry (ZooMS) which identifies animal species by means of characteristic peptide sequences in the protein collagen, for example in vellum.) The collagen of the filament may be subjected to extraction, denaturation, digestion and filtration, followed by mass spectrometric analysis. Extraction of collagen into solution may be accomplished using an ammonium bicarbonate buffer. Denaturation may then be accomplished by heating the solubilised sample to a temperature of about 65 °C. Trypsin may then be added to cleave collagen after each arginine or lysine amino acid in its sequence, resulting in peptide fragments of predictable masses. After digestion, the sample may be filtered with C18 filters to remove non-collagenous material. The sample may then undergo mass spectrometric analysis, such as by MALDI-TOF MS.

Additionally or alternatively, the collagen filament may be identified or characterised using x-ray crystallography, which may provide a measure for the extent of crystallinity of the collagen. The collagen molecules may exhibit a level or type of crystallinity characteristic of a filament produced by the method of the first or third aspect of the disclosure.

Additionally or alternatively, the filament may be identified or characterised by spectrometry or spectroscopy to identify trace impurities therein. This may enable characterisation of the source of the collagen, for example that it derives from lime split; and/or that it underwent the method according to the first aspect of the invention, for example due to the presence of cross-linking and/or trace ions.

The collagenous filament may especially have a diameter in a range of from about 40 to about 500 μm, more especially of from about 80 to about 250 μm.

The collagenous filament is, by definition, longer than it is wide. The length of the collagenous filament may be hundreds, thousands or millions times its diameter. It may typically have a length of about 500 mm or more, for example tens of thousands of metres, e.g. at least 10,000 metres.

The collagenous filament may have a tenacity (the amount of force needed to break it, divided by the linear mass density of the thread) of about 0.1 or more grams per denier (optionally, up to about 10 grams per denier), especially about 0.5 or more grams per denier (optionally, up to about 7 grams per denier), most especially about 4 grams per denier. Tenacity may be measured in accordance with ASTM D2256.

The collagenous filament may be subjected to further chemical processing, for example using one or more pigment(s) and dye(s). It may be subjected to processing with fatliquors. It may be subjected to processing with softening agents and/or secondary plasticizers. It may be further cross-linked, for example using cross-linkers that act on other parts of the collagen molecule than lysine residues. It may be processed to be hydrophobic. It may be processed to be flame retardant. The collagenous filament is of a nature to be compatible with all these and other chemical processing steps.

The collagenous filament may be subjected to further mechanical processing, such as crimping or other texturing, thereby increasing cohesiveness (propensity to cling together), bulk and warmth. The collagenous filament is of a nature to be compatible with all these and other mechanical processing steps.

As used herein, the term “monofilament yarn” refers to yarn consisting of a single filament, such as yarn consisting of the collagenous filament of the fourth aspect of the present disclosure. In contrast, the term staple yarn refers to yarn consisting of short-length fibers bundled together.

The collagenous filament according to all aspects of the disclosure may be used as, thus may become or be, a monofilament yarn.

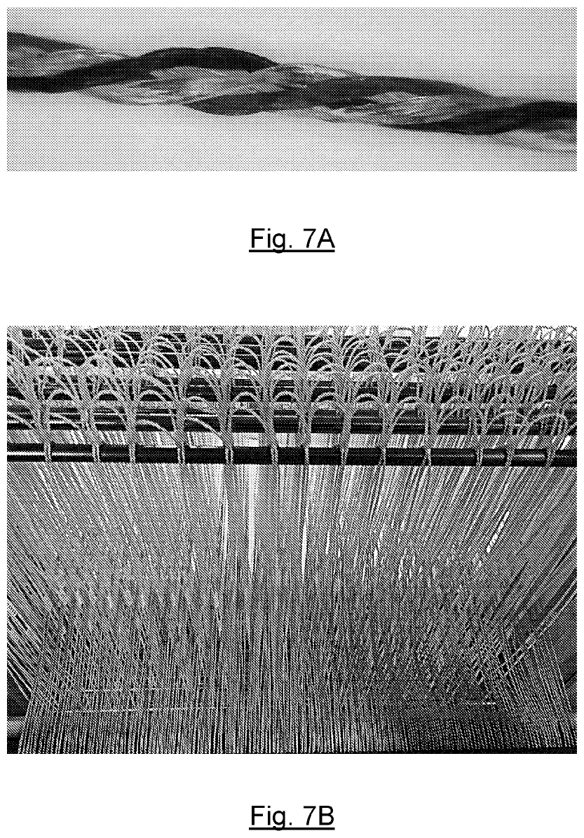

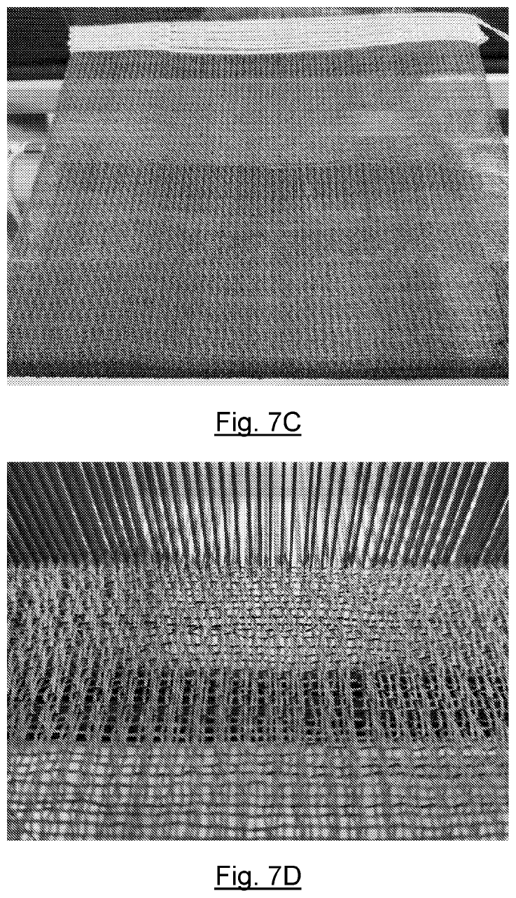

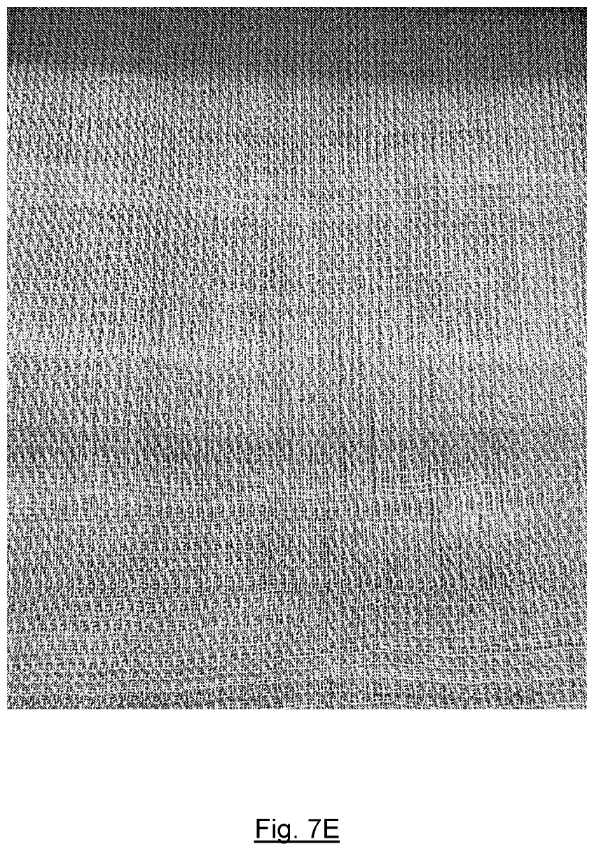

Multifilament yarns include single yarns, comprising two or more monofilament yarns twisted together; ply yarns, comprising two or more single yarns twisted together; and cord yarns, comprising two or more ply yarns twisted together. A multifilament yarn may comprise the collagenous filament of the fourth aspect of the present disclosure. The collagenous filament may be twisted therein with one or more further such collagenous filaments and/or one or more non-collagenous filaments and/or one or more staple yarns. FIG. 7A is a photograph of a multifilament yarn consisting of collagenous filaments in accordance with the fourth aspect of the present disclosure.

In a fifth aspect, there is provided an article comprising at least one collagenous filament defined in accordance with the fourth aspect of the disclosure. Optionally, the article is a textile. The textile may be a woven or knitted textile. Optionally, the textile is for footwear or for apparel.

FIG. 7B is a photograph of the multifilament yarn of FIG. 7A loaded onto a loom in preparation for weaving. FIG. 7C is a photograph of a textile being woven on the loom from the multifilament yarn. FIG. 7D is a magnified section of FIG. 7C. FIG. 7E is a photograph of the woven textile once finished and removed from the loom. Without wishing to be bound by theory, it is thought that the tenacity of the collagenous filament enables weaving of the yarn without undue breakage.

Preferably, the textile is a woven or knit synthetic leather. Thus, the present disclosure may provide for taking waste hides, such as low value hides (e.g. due to scarring of cattle hides during the winter months or by contact with barbed wire) and upcycling them into soft, low-blemish or blemish-free leather of higher value, for use in, for example, footwear, such as footwear uppers, tongues and the like.

Optionally, the article is an article or component of footwear.

Preferably, the article is a (2D and/or 3D) knit footwear upper. The upper most preferably comprises a single filament yarn or a multifilament yarn comprising a plurality of collagenous filaments defined in accordance with the fourth aspect of the disclosure.

Optionally, the article is an article or component of apparel.

Optionally, the article is an article that would otherwise be a leather good. Thus, optionally, the article is an article or component of a bag, suitcase or upholstery.

It will be appreciated that features described in relation to one aspect of the present disclosure may be incorporated into other aspects of the present disclosure.

Preferably, in all aspects of the present disclosure, the collagen is waste collagen.

Significant quantities of collagen are found in animal hide (also known in the art as a pelt or skin). One or more of bovine, ovine, caprine and porcine (especially, bovine) hides may be sources of the collagen of the present disclosure. It will be appreciated that leather is not hide; see, for example, ISO 15115:2019.

The collagen of the present disclosure may derive from by-products of industry, especially low-value by-products, such as by-products of slaughterhouses and tanneries. Often, low value animal hides (such as those of cattle which have survived extreme conditions, for example in the winter months, or cattle exposed to barbed wire, and which are therefore badly damaged) are sent to landfill, burnt, buried or otherwise disposed of as being unsuitable for processing into finished leather.

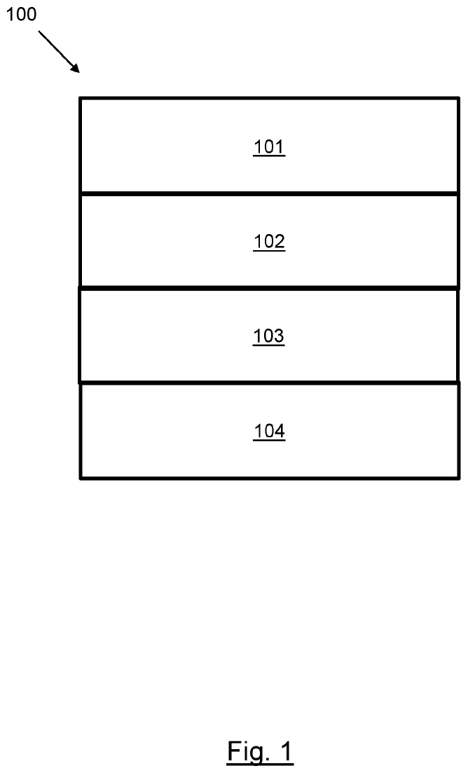

As shown schematically in FIG. 1, the hide 100 of an animal comprises a corium layer 103. The corium 103 may provide a rich source of collagen. The corium 103 may be abutted, on its external side, by a grain layer 101. In FIG. 1, the junction between the corium and the grain layer is also shown as 102. On its other, internal side, corium 103 is abutted by flesh 104.

Preferably, in all aspects of the present disclosure, the collagen is collagen of one or more animal hides (especially bovine hide). Especially, the collagen may be of the corium of the one or more animal hides.

In the tanning industry, a “split” may be or comprise the bottom strata of the corium, separated from the remainder of the hide using a splitting machine, e.g. a band knife splitting machine. By passing a (typically, part-processed, such, for example, as limed) hide through a (band knife) splitting machine, two strata are obtained: the split (i.e. layer of the corium nearest to the flesh side) and the grain split (that also contains a significant proportion of the corium). Splitting a limed hide results in the production of a lime split, a term well known in the art. See, for example, ISO 15115:2019.

The collagen of the present disclosure may be collagen of lime split or lime split trimmings. An advantage of lime split and lime split trimmings is a relatively low level of impurities, and a relatively high level of collagen.

Although the collagen of the present disclosure may be derived synthetically, for example from cell cultures or other laboratory sources of collagen, such as where collagen is “grown” on a suitable scaffold (e.g. on a polymeric mesh), this may be a slower and more expensive way to obtain collagen. While these sources, having assured purity, may be useful for collagen for biomedical applications, they may be more expensive or slower to generate collagen.

### DETAILED DESCRIPTION

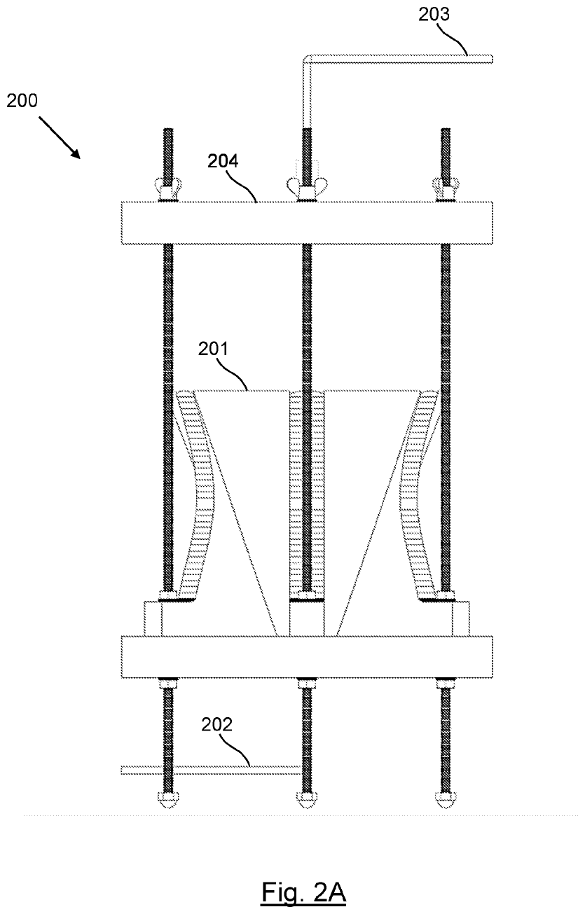

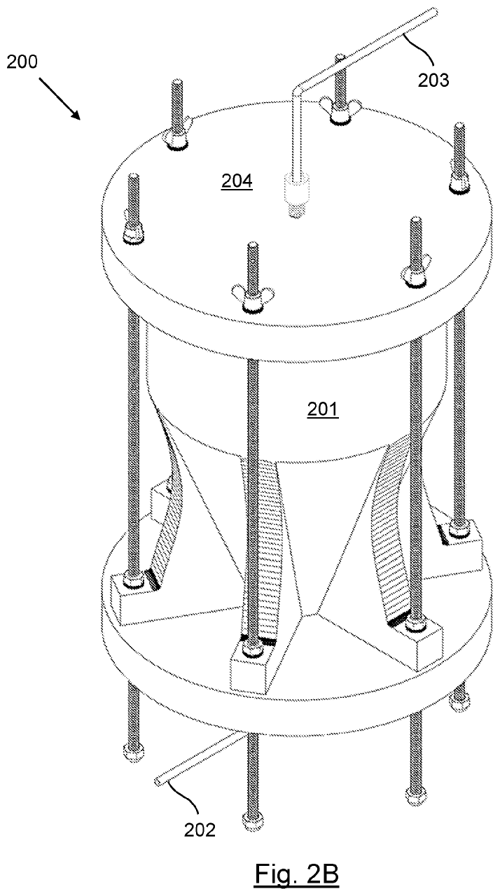

In FIG. 2A, an extrusion pressure vessel 200 is shown, comprising airtight tubing 203 for feeding air under positive pressure. Tubing 203 is connected via an airtight seal to the top 204 of a container 201 having a substantially frustoconical portion. During use, the container contains a solution of collagen in water (not shown) at a temperature of about 3 to about 4 °C. Temperature control may be achieved by housing extrusion pressure vessel 200 in a temperature control chamber (not shown). At the bottom of container 201 is an airtight seal to airtight tubing 202 for feeding a solution of collagen in water to an extrusion needle (not shown). FIG. 2B shows a perspective view of the extrusion pressure 200 vessel of FIG. 2A. The same reference numerals are used for the same components in FIG. 2B as in FIG. 2A.

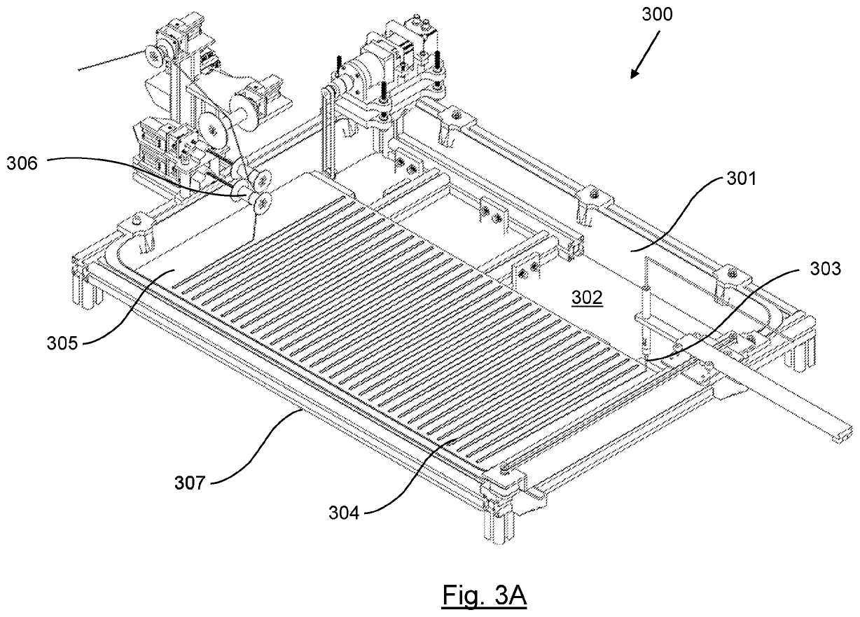

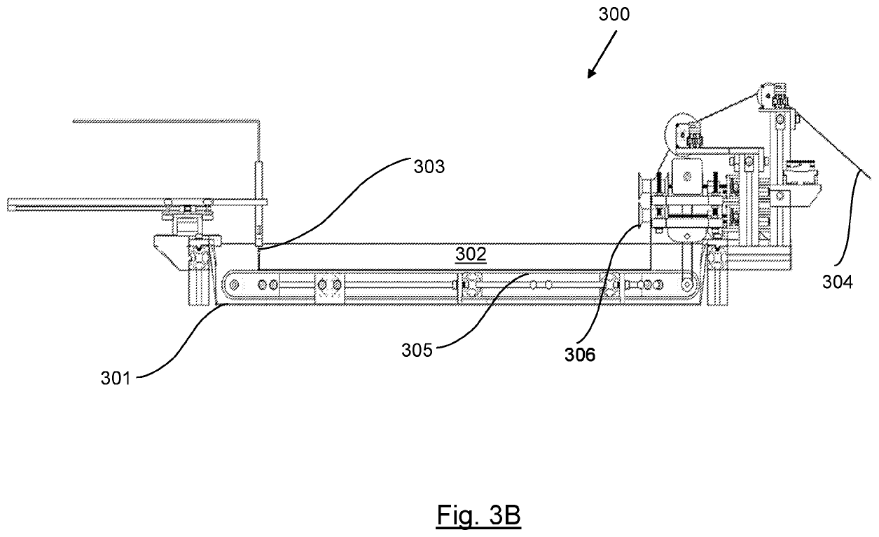

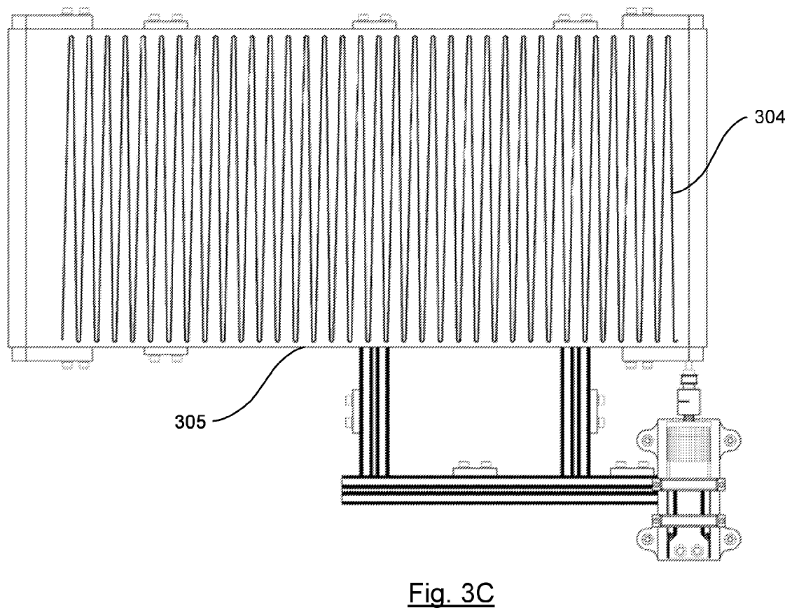

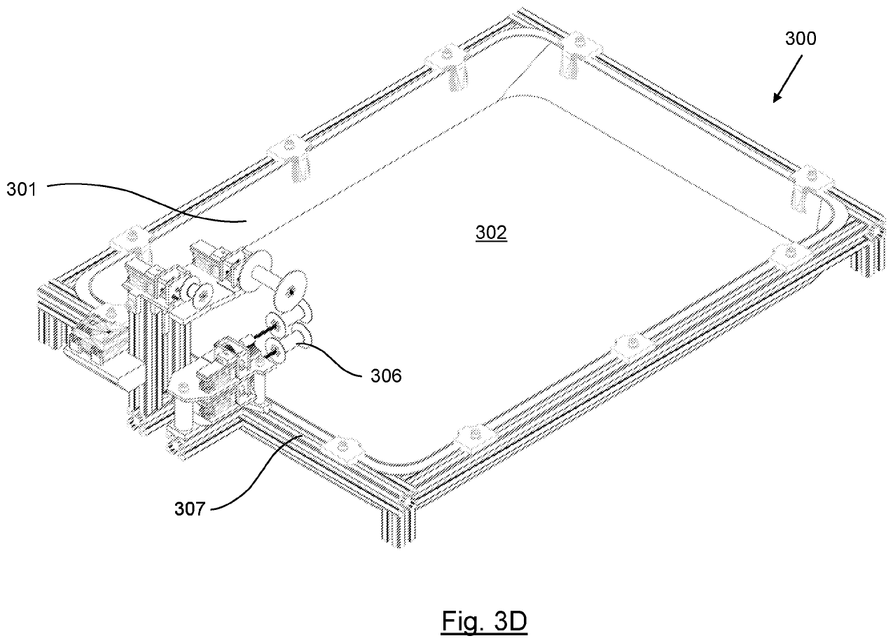

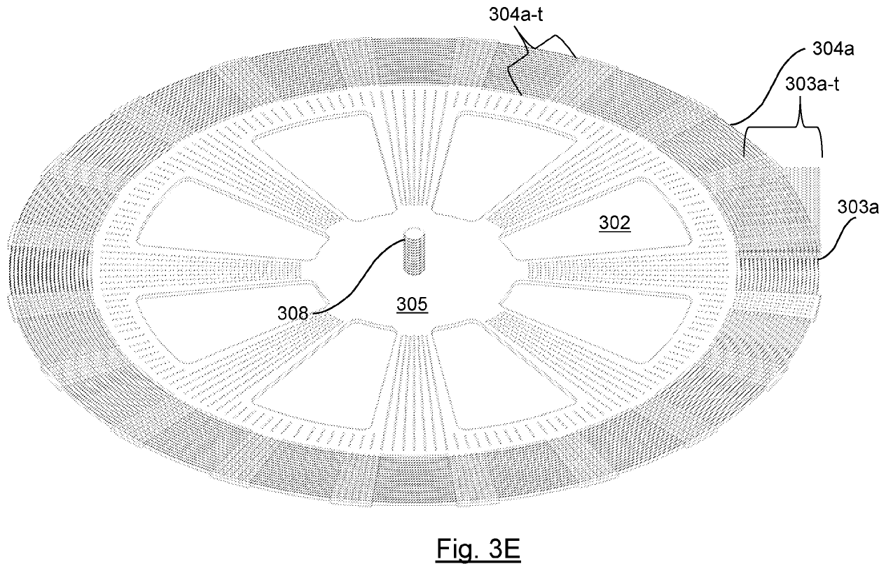

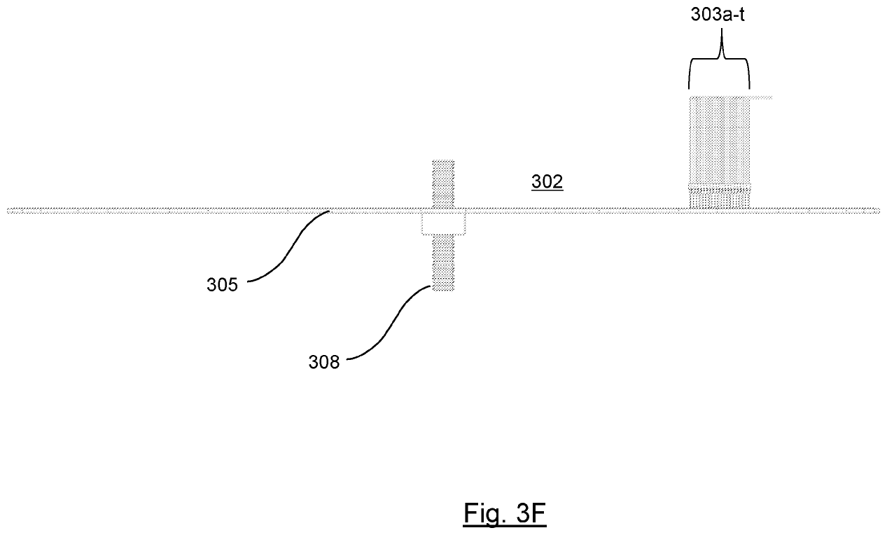

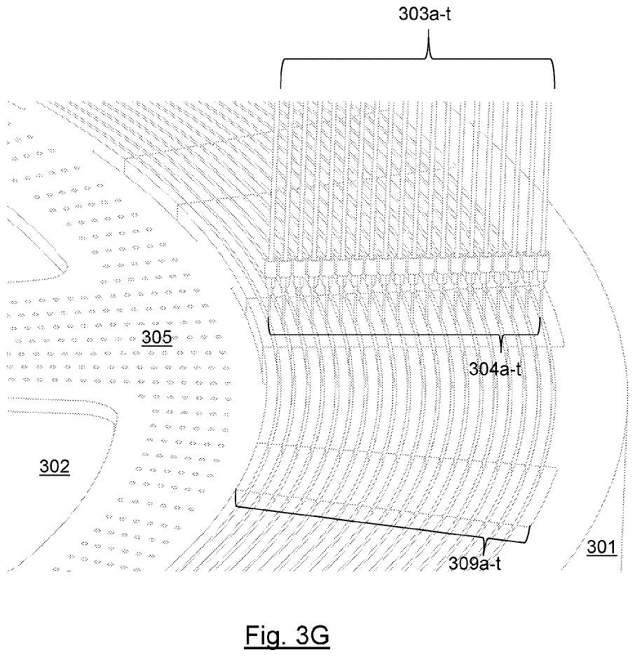

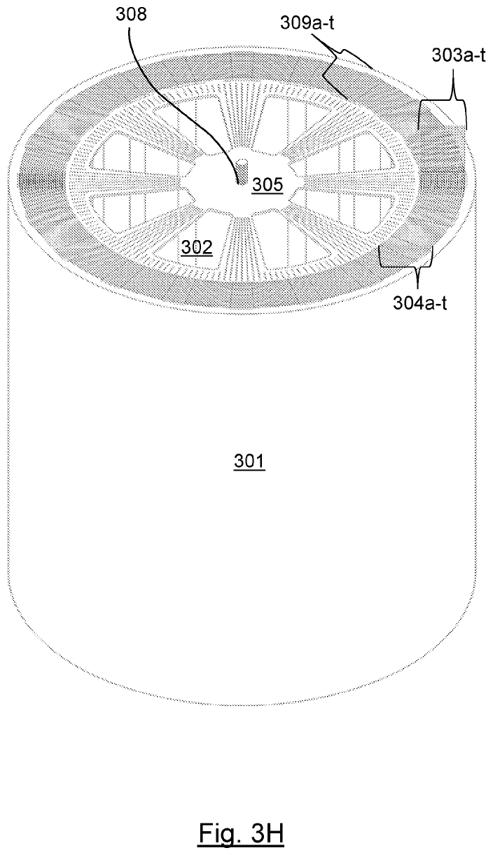

In FIG. 3A fibrillation bath 300 is shown having a fibrillation bath vessel 301 containing, in use, fibrillation bath liquid 302. Extrusion needle 303 extrudes a continuous thread of collagenous extrudate 304 into the liquid 302 and onto the surface of build platform 305. The surface of the build platform 305 moves, in use, and supports the thread 304 through the liquid 302 as it does so. In FIG. 3A, the build platform 305 is a conveyor belt. Build platform 305 could have another form, provided that, in use, it is horizontal and moves and supports the thread 304 through the liquid 302. Once thread 304 has been moved through the liquid 302 for about 1 hour, it comes to an end of the bath 300 where it is picked up by a first take-off roller 306 in a series of take-off rollers, for transferring thread 304 to a drying module (not shown). The components shown in FIG. 3A are supported by a frame 307. FIG. 3B shows a side cross-sectional view of what is shown in FIG. 3A, using the same reference numerals. Similarly, FIG. 3C shows a top view of the moveable build platform 305 of FIG. 3A. FIG. 3D shows a perspective view of the bath 300 and take-off rollers of FIG. 3A, along with the frame 307, but without build platform 305.

FIG. 3E shows a type of build platform other than a conveyor belt. A plurality of extrusion needles 303a-t each extrude a respective thread 304a-t towards the surface of build platform 305. For example, extrusion needle 303a extrudes thread 304a (both labelled individually). Build platform 305, which is horizontal relative to surrounding fibrillation bath liquid 302, has a perforated surface, meaning it can move vertically relative to the bath liquid 302 and the liquid 302 can pass through it. That vertical movement is facilitated by lead screw 308, although it could be achieved using another element having the same function of being able to move the build platform 305 vertically relative to the liquid 302. FIG. 3F shows a side view with the same reference numerals as in FIG. 3E. FIG. 3G shows a close-up view of tubing and extrusion needles 303a-t extruding threads 304a-t towards the surface of moveable build platform 305. FIG. 3G shows how guide channels 309a-t guide threads 304a-t towards the surface of moveable build platform 305. Guide channels 309a-t are coupled to a fibrillation bath vessel 301. In use, build platform 305 slots into guide channels 309a-t. By using guide channels 309a-t, any given thread 304a-t is prevented from contacting threads extruded from extrusion nozzles adjacent to its own extrusion nozzle, and is guided into precisely the correct position supported by the surface of build platform 305.

As will be appreciated from FIG. 3E, the surface of build platform 305 has a substantially circular shape. In use, the surface of build platform 305 translates down the shaft of lead screw 308. First lengths of threads 304a-t are extruded by extrusion needles 303a-t in a circular path onto the surface of build platform 305. Enough time (e.g., about 3 seconds) passes for some fibrillation of the external surface of the first lengths of threads 304a-t to occur. The first lengths of threads 304a-t are then stable enough for second lengths of the same, continuous threads to be extruded towards them (so that first and second lengths of threads 304a-t are supported by the underlying surface of build platform 305) as the surface of build platform 305 translates back up the shaft of lead screw 308. In this way, layered coils of threads 303a-t can be stably formed, all supported by the surface of build platform 305, before being transferred to respective drying modules. FIG. 3H shows an entire cylindrical fibrillation bath vessel 301 with build platform 305 towards the top end of lead screw 308. It will be appreciated that other (e.g., polygonal) shapes could be used.

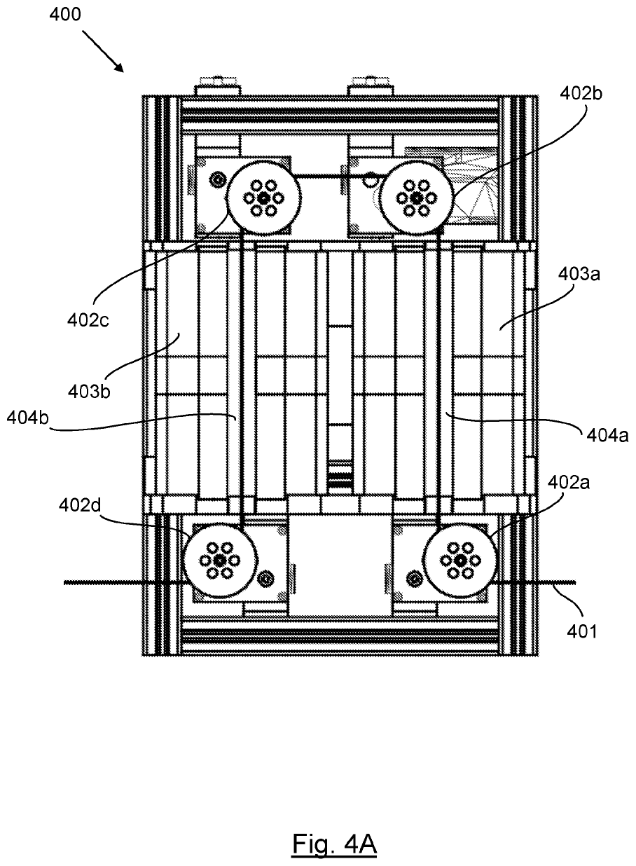

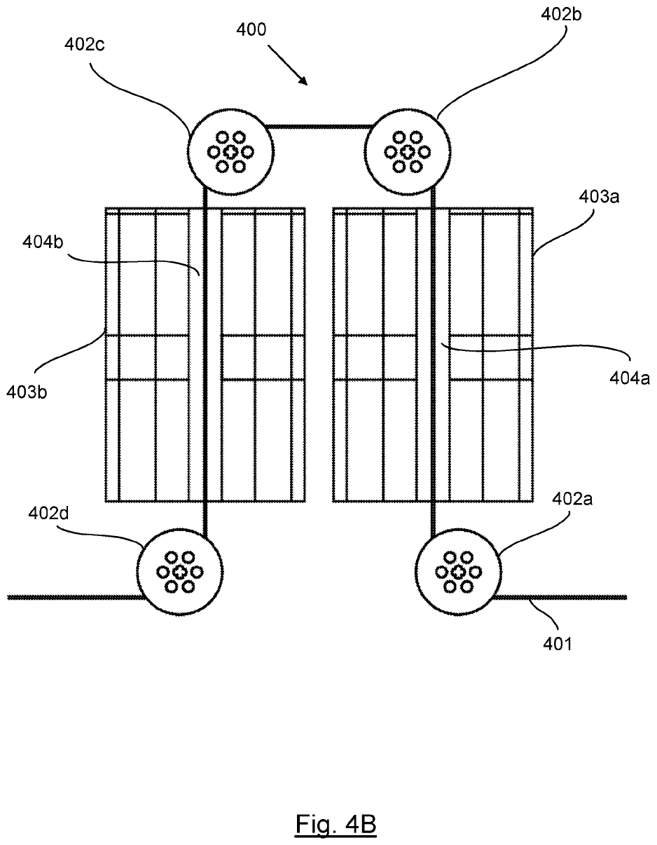

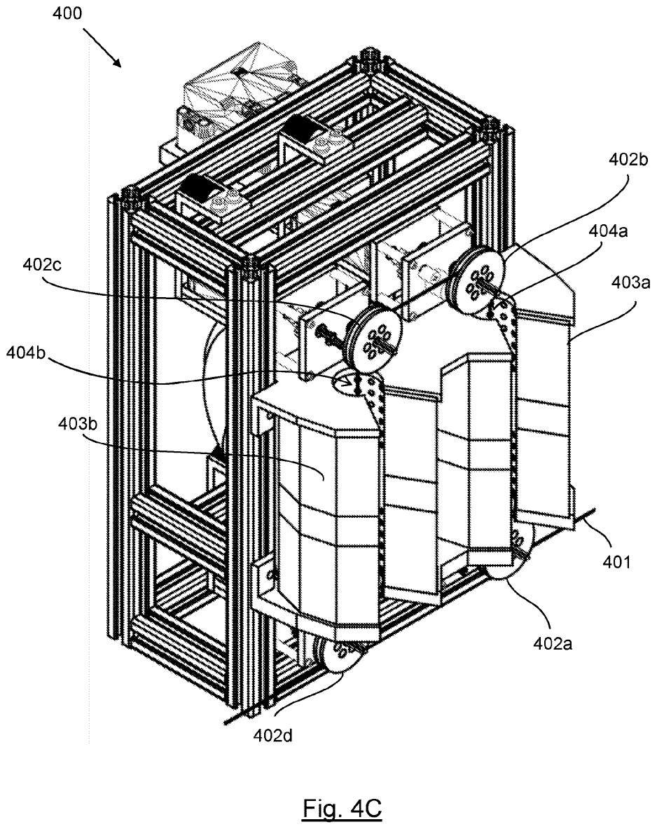

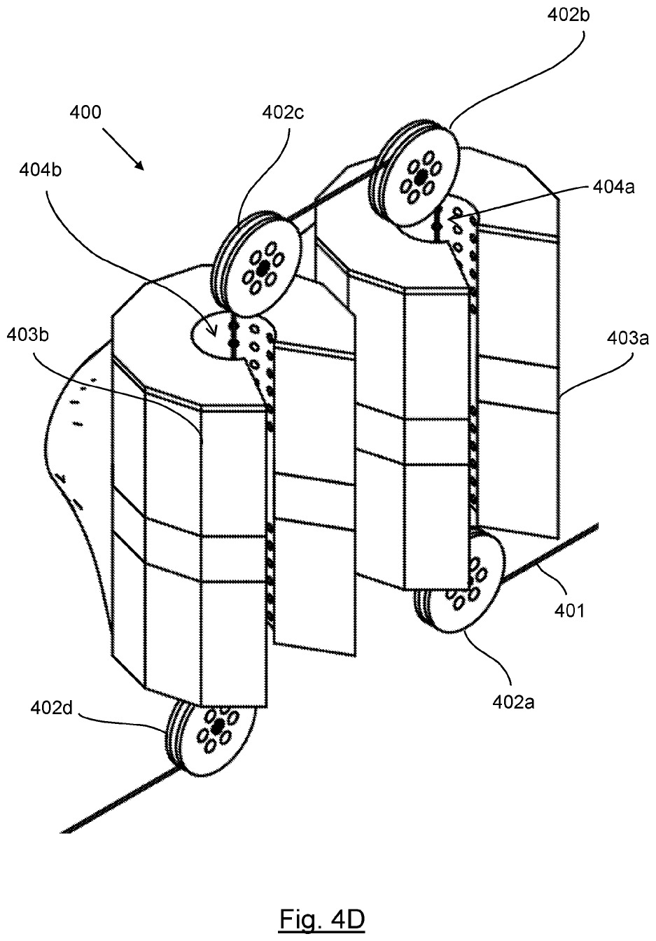

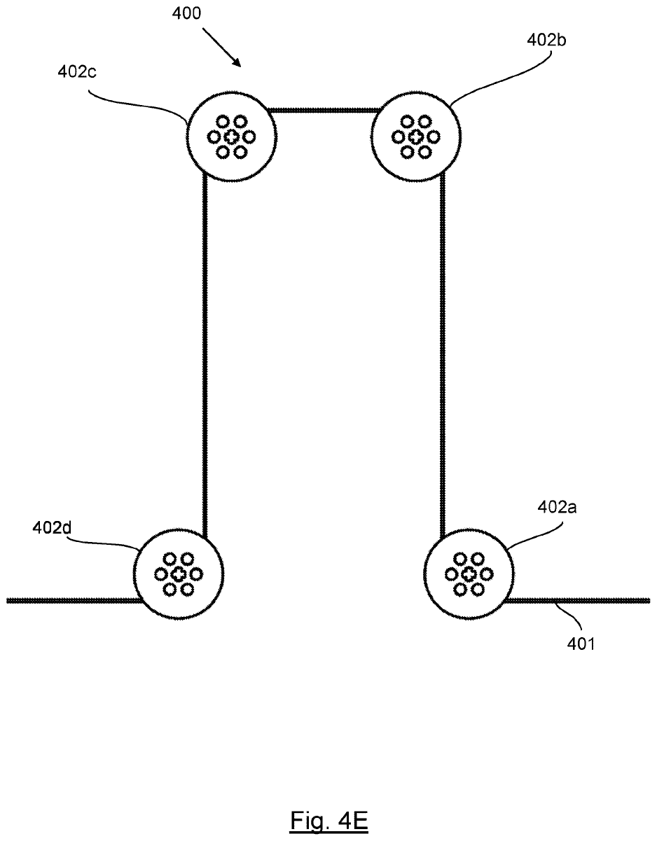

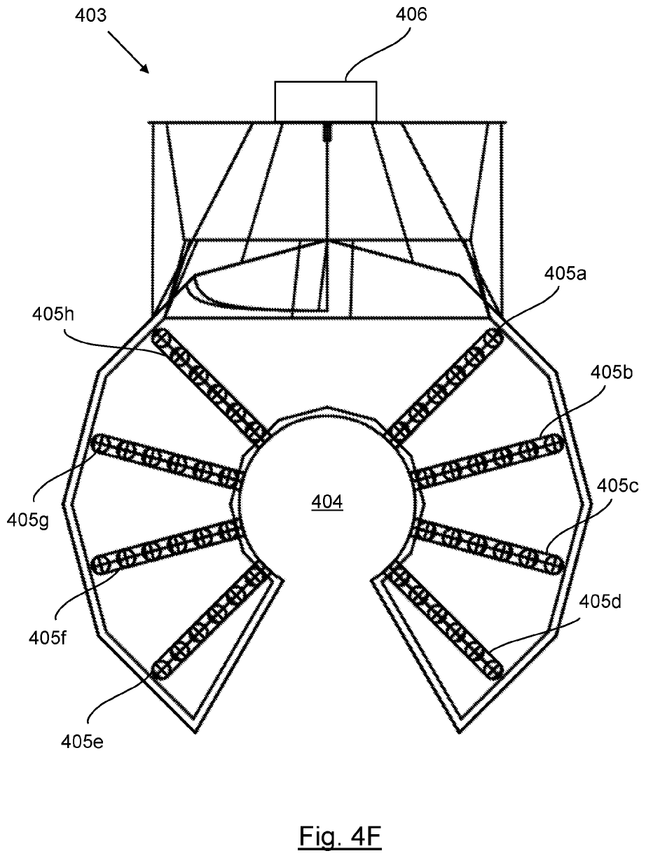

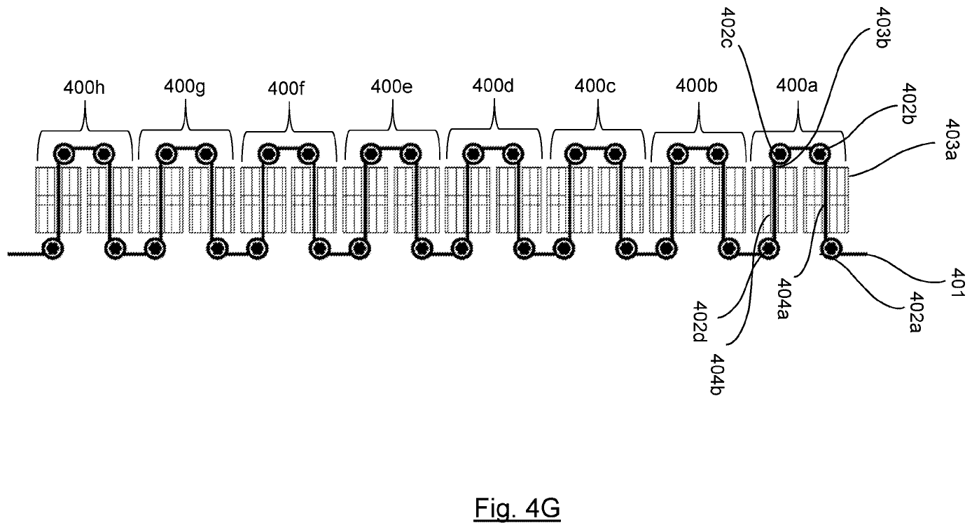

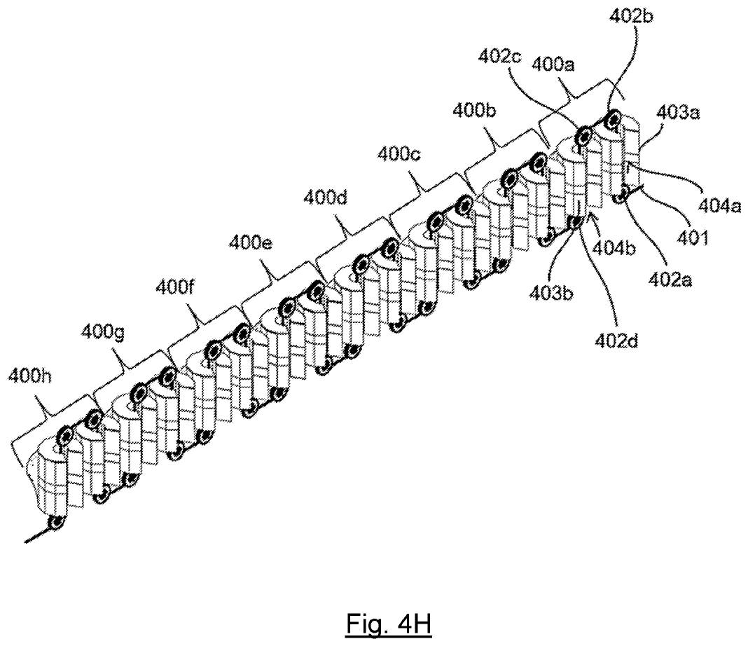

FIG. 4A shows a drying module 400. A continuous thread of collagenous extrudate 401 enters the drying module and passes over roller 402a into drying cavity 404a which is warmed by heater 403a surrounding the drying cavity. Thread 401 then moves over roller 402b to roller 402c spaced apart from roller 402b. Thread 401 passes over roller 402c into drying cavity 404b which is warmed by heater 403b surrounding drying cavity 404b. Thread 401 then passes over roller 402d and out of the drying module 400. From drying module 400, thread 401 can move to a further drying module (not shown) or to a winder (not shown). FIG. 4B shows a simplified side view of drying module 400. FIG. 4C shows a perspective view of drying module 400. FIG. 4D shows a simplified perspective view of the drying module 400. FIG. 4E shows a further-simplified side view of the drying module of FIG. 4A. The same reference numerals are used for the same components in FIGS. 4B to 4E as in FIG. 4A.

FIG. 4F shows a heater 403 with a drying cavity 404. The heater 403 comprises a plurality of air channels 405a-h part-encircling the drying cavity 404. Each air channel 405a-h opposes another air channel 405a-h situated across the drying cavity 404. In use, air is heated and fed through the air channels 405a-h at a low speed, for example about 0.8 m/s. The provision of equal and opposing air channels 405a-h, part-encircling the drying cavity 404 (and thus, in use, the thread, which is not shown) enables hot air within the drying cavity 404 to have a net velocity of about 0 m/s to reduce vibrational and/or sideways motion of thread as it dries. A diffuser 406 makes the air flow gentler.

FIG. 4G shows a simplified side view of a plurality of drying modules of FIG. 4A arranged in series. The same reference numerals are used for the same components in FIG. 4G as in FIG. 4A. Meanwhile, FIG. 4H shows a simplified perspective view the plurality of drying modules. FIG. 4I shows a further-simplified side view of a plurality of drying modules of FIG. 4A arranged in series (i.e., heaters not shown). Only one drying module 400a is provided with a complete set of reference numerals in FIGS. 4G to 4I, but it will be appreciated that drying modules 400b-h have the same features as drying module 400a.

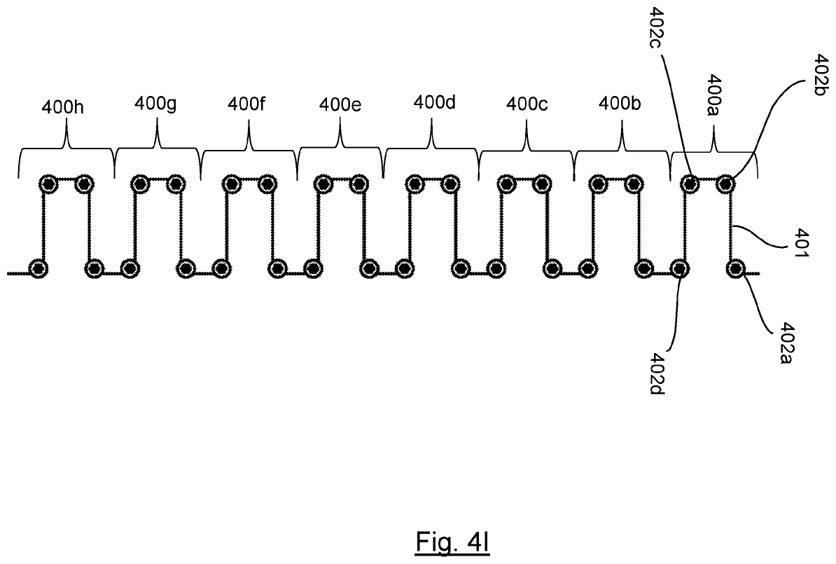

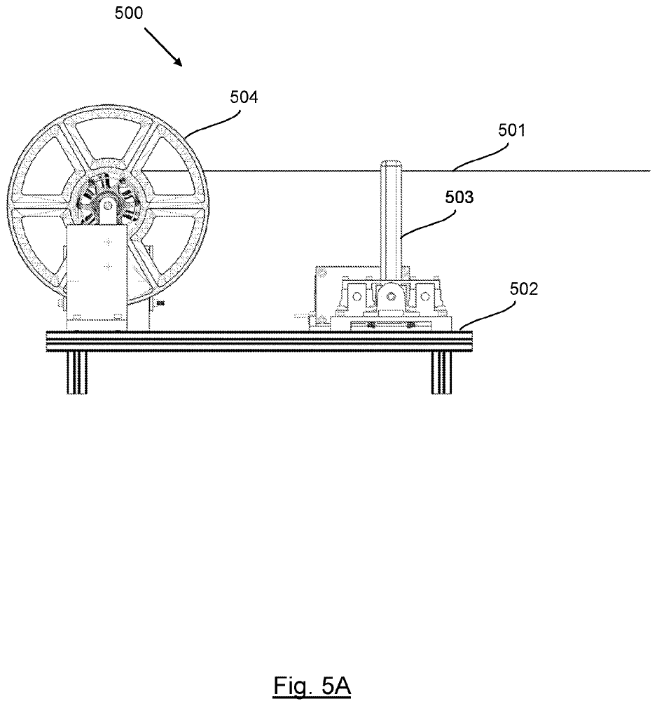

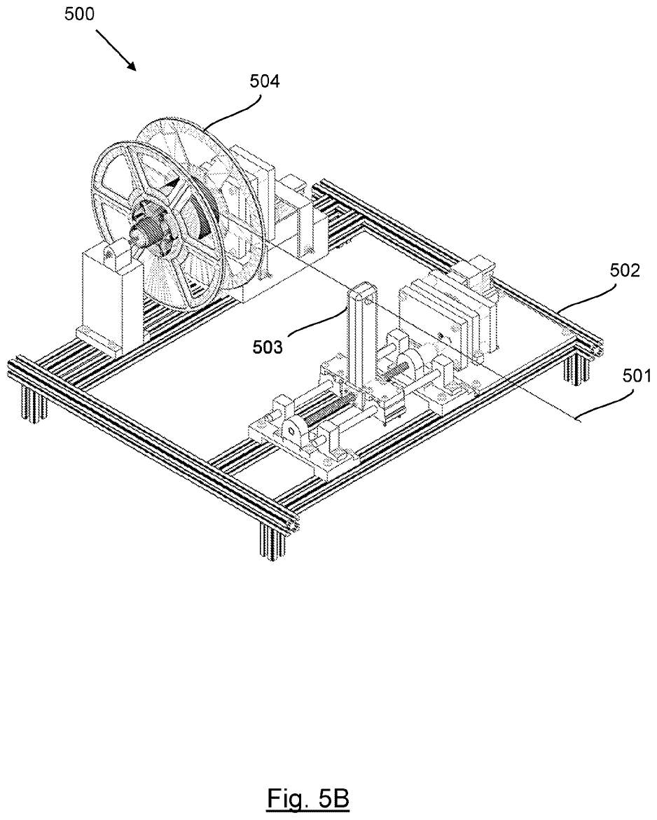

FIG. 5A shows a side view of a winder 500. Collagenous textile filament 501 is fed through feeder 503 to winding roller 504 (e.g., a spool, as shown in FIG. 5A). Winding roller 504 is supported, as is feeder 503, on support frame 502. FIG. 5B shows a perspective view of the winder of FIG. 5A. The same reference numerals are used for the same components in FIG. 5B as in FIG. 5A.

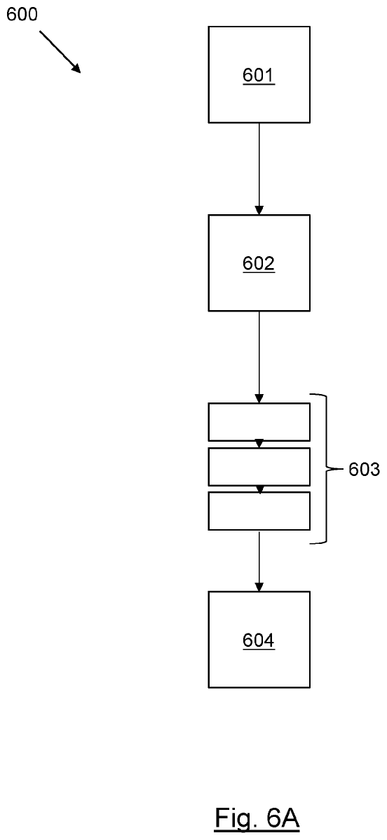

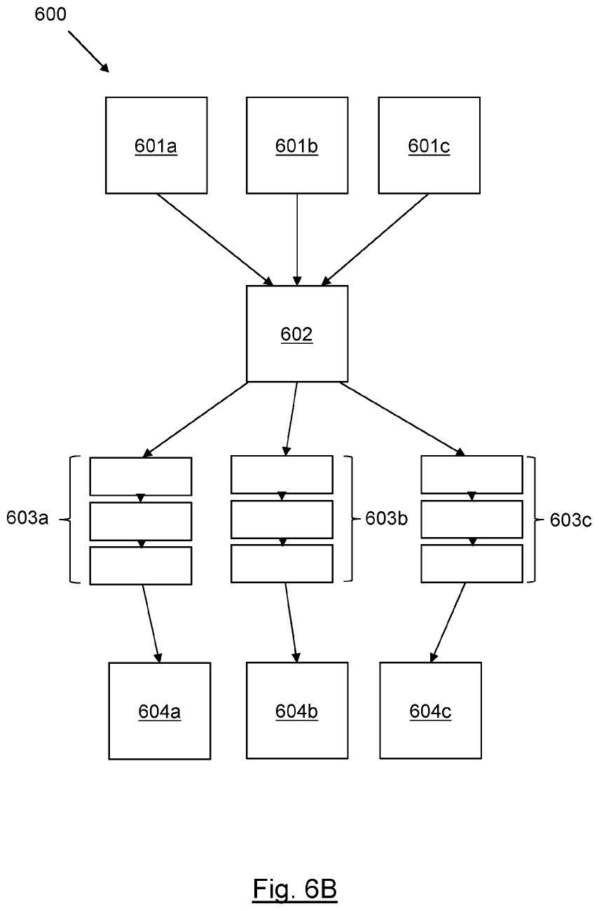

Shown schematically in FIG. 6A is apparatus 600 for forming a collagenous textile filament. The apparatus comprises extrusion vessel 601, containing a solution of collagen in water and connected to an extrusion needle via airtight tubing (not shown). In use, the extrusion needle (also referred to as an extrusion nozzle) extrudes a continuous thread of collagenous extrudate into the liquid of fibrillation bath 602 and onto the surface of a moveable build platform (not shown) contained in the fibrillation bath vessel of bath 602. The liquid of fibrillation bath 602 is an aqueous fibrillation and tanning solution. Thread is transferred via take-off rollers (not shown) from bath 602 to a plurality of drying modules 603. Although three drying modules are present in FIG. 6A, it will be appreciated that there may be more than three, for example there may be 8 drying modules. Thread is then moved through each drying module in series in the plurality of drying modules 603. The thus-formed collagenous textile filament is transferred to winder 604 for winding. In the apparatus 600 of FIG. 6A, there are no intermediary baths between bath 602 and the drying modules 603.

Shown schematically in FIG. 6B is apparatus 600 for forming a plurality of collagenous textile filaments. The apparatus comprises extrusion vessels 601a-c. Although three extrusion vessels (with three corresponding extrusion needles and tubing, not shown) are present in FIG. 6B, it will be appreciated that there may be more than three, for example there may be 20 extrusion vessels (and 20 extrusion needles) for forming 20 threads of collagenous extrudate. In use, continuous threads of collagenous extrudate are extruded into the liquid of fibrillation bath 602 and towards the surface of a moveable build platform (not shown) contained in the fibrillation bath vessel of bath 602 (i.e., all of the plurality of threads are supported by the same surface of the same moveable build platform). The liquid of fibrillation bath 602 is an aqueous fibrillation and tanning solution. The plurality of threads are supported upon the moving build platform surface through the liquid of the bath. Then, each thread of collagenous extrudate is transferred via take-off rollers (not shown) to its respective plurality of drying modules 603a-c. Each thread is then moved through each drying module in its respective plurality of drying modules 603a-c. Although three drying modules are present in each plurality of drying modules 603a-c of FIG. 6B, it will be appreciated that there may be more than three, for example there may be 8 drying modules in each plurality of drying modules 603a-c. The thus-formed collagenous textile filaments are transferred to winders 604a-c for winding. In the apparatus 600 of FIG. 6A, there are no intermediary baths between bath 602 and the pluralities of drying modules 603a-c.

### EXAMPLES

### Example 1—Preparation of 5% w/w Collagen Stock Solution

The purpose of Example 1 is to prepare a solution of collagen in water, having a concentration of collagen of 5% w/w. The starting material is lime split, obtained as a tannery by-product (lime split may be produced by chilling flayed hide, soaking, unhairing and liming it, lime fleshing it, and lime splitting it, as is known in the art; see, for example, Azdet, JALCA, Vol. 105, 2010, the entire contents of which are hereby incorporated herein). Example 1 thus involves the removal from lime split of non-collagenous material.

The obtained 5% w/w collagen solution can be stored for extended periods of time at or below about 4 °C. Maintaining the stock solution at or below that temperature is thought to help avoid thermal destabilisation of the collagen (dissociation or “melting” of the triple helix into polypeptide chains).

### Apparatus

Leather Processing Drums
Phenolphthalein Indicator Solution
Bromocresol Green Indicator Solution
Mincer with Specific Aperture Size Plates
Fridge/Cold Storage/Ice Maker as necessary
Vacuum Blender/Homogeniser
Moisture Balance/Oven
Balance Scales

### Method

Obtain lime split (e.g., from a tannery). Record its mass, then load it into drums and perform the following steps.
i. Closed wash—150% water (i.e. 1.5×the mass of the split) at 30 °C for 20 minutes
ii. Drain drum
iii. Closed wash—150% water (i.e. 1.5×the mass of the split) at 30 °C for 20 minutes
iv. Drain drum
v. Re-float with 100% water (i.e. 1×the mass of the split) at 35 °C
vi. Add 0.5% ammonium chloride, run for 20 minutes
vii. Add 0.5% ammonium chloride, run for 90 minutes

Steps (vi.) and (vii.) assist with the de-liming of the raw split material. Non-ammonium chloride-based de-liming steps, known in the art, will also be satisfactory; e.g., CO₂ de-liming; dicarboxylic acid deliming; etc. These could replace the above-mentioned ammonium chloride processing.
viii. Check pH, which should be in a range of from 8.0 to 8.5. If outside range, adjust as necessary ammonium chloride. (Note: this can be altered as required depending on the subsequent bating product to be used)
ix. Check pH of cross-section of split using phenolphthalein pH indicator solution (result should be colourless). If still pink, then run on as necessary and recheck after an additional 30 minutes.
x. Drain drum
xi. Re-float with 50% water at 30 °C
xii. Add 0.5% Eusapon OC (a non-ionic degreasing agent, obtainable from Stahl Holdings B.V., Sluisweg 10 5145 PE Waalwijk, Netherlands)
xiii. Optionally, add a sequestering agent (for the removal of residual bound calcium ions)
xiv. Run drum for 3 hours
xv. Drain drum
xvi. Closed wash—150% water (i.e. 1.5×the mass of the split) at 30 °C for 20 minutes
xvii. Drain drum
xviii. Closed wash—150% water (i.e. 1.5×the mass of the split) at 30 °C for 20 minutes
xix. Drain drum
xx. Closed wash—150% water (i.e. 1.5×the mass of the split) at 30 °C for 20 minutes
xxi. Drain drum
xxii. Re-float with 100% water @ 30 °C
xxiii. Add 2% w/w sodium citrate and 2% citric acid w/w as a sequestering buffer system that will give a consistent pH and help to remove any final bound calcium ions
xxiv. Run drum for 1 hour
xxv. Check pH, which should be in a range of from 4.2-4.5. If outside range, adjust as necessary with sodium citrate and/or citric acid
xxvi. When pH is correct, continue running drum for 5 hours
xxvii. Check pH of cross-section of splits with bromocresol green pH indicator solution (result should be yellow-light green). If incorrect (i.e. a blue colour which indicates a higher pH) recheck pH of liquor and adjust with citric acid and run on drum as necessary
xxviii. Drain drum
xxix. Closed wash—150% water (preferably deionised water) (i.e. 1.5×the mass of the split) at 30 °C for 20 minutes
xxx. Drain drum
xxxi. Closed wash—150% water (preferably deionised water) (i.e. 1.5×the mass of the split) at 30 °C for 20 minutes
xxxii. Drain drum
xxxiii. Closed wash—150% water (preferably deionised water) (i.e. 1.5×the mass of the split) at 30 °C for 20 minutes
xxxiv. Drain drum
xxxv. Closed wash—150% water (preferably deionised water) (i.e. 1.5×the mass of the split) at 30 °C for 20 minutes
xxxvi. Remove splits from drum
xxxvii. Sammy splits to remove excess water (by squeezing between the rollers of a sammying machine)
xxxviii. Fold splits on to pallets
xxxix. Optionally, store the splits at a temperature in a range of from 2-4 °C for up to several days, such as up to a week
xl. Cut split material into strips whilst keeping temperature below 4 °C.

Cutting the split material may be automated, for example using a through feed water-jet cutter, provided there is little or no heat build-up at the cutting surface, which would may otherwise lead to localised denaturation of the collagen.
xli. Feed strips into meat mincer with 16 mm diameter drilled plate ×3, then cool to below 4 °C. Optionally, the equipment could combine this and the next three steps simultaneously, using a refrigerated cooling jacket to ensure temperatures remain low.
xlii. Feed through mincer with 10 mm diameter drilled plate ×3, then cool to below 4 °C.
xliii. Feed through mincer with 8 mm diameter drilled plate ×3, then cool to below 4 °C.
xliv. Feed through mincer with 4.5 mm diameter drilled plate ×3 times, then cool to below 4 °C.
xlv. Store minced collagen at a temperature in a range of from 2-4 °C.
xlvi. Take three samples of minced collagen to determine the % by weight solids content (M_{sc}). This can be achieved in a variety of ways, essentially drying the material fully (established by the weight reducing to a stable point). Such techniques include moisture balances, simplistic oven methods, etc. Typical minced collagen sample sizes may be 50 g. More samples can be taken to improve accuracy. It is important to spread the mince out on a sample holder to allow all moisture to escape and improve accuracy of the data.
xlvii. It is then possible to determine the quantity of cold (4 °C) 0.01M hydrochloric acid (HCl) to be added to create a 5% gel stock, using the following formula:

Mass of 0.01M HCl required (in grams)=((Mₘ×M_{sc})/5%)
where Mₘ=mass of the mince to be used (in grams) and M_{sc}=The solids content of the mince (in % by weight).

After the mincing stage, it is advisable to avoid the formation of air bubbles. Air bubbles present in the collagen solution may cause discontinuities in downstream collagen extrudate.
xlviii. Place mince into a temperature controlled vacuum homogeniser (for small scale laboratory work a vacuum blender can be used). Optionally, a through-feed re-circulatory chilled vacuum homogeniser may be used.
xlix. Add the calculated amount of chilled 0.01M HCl. Ensure the temperature is maintained at lower than 10 °C.
l. Homogenise for at least five minutes in the homogeniser.
li. Observe the collagen solution. At completion, it should appear as a viscous (almost solid) gel that has a slightly cream/yellow tinge to it.
lii. Re-chill the 5% w/w collagen solution. It is capable of being stored at 4 °C for an extended time period, e.g. a week.

### Example 2—Preparation of 1.25% w/w Collagen Dope for Extrusion (From 5% w/w Stock)

5% w/w aqueous collagen solution may be viscous (without wishing to be bound by theory, it is thought that this is due to the hydrophilicity of collagen). 5% w/w aqueous collagen solution may be too viscous to extrude into fibres at small diameters; although if this is desired, it is possible using very high pressures.

Example 2 is to provide 1.25% w/w aqueous collagen solution for extrusion. The 1.25% w/w aqueous collagen solution is also referred to as collagen dope.

It is advantageous to avoid or mitigate the formation of air bubbles in the aqueous collagen solution, as they may otherwise form discontinuities during extrusion.

### Apparatus

Fridge (or other cold storage)
Vacuum blender/homogeniser
Moisture balance/oven
Vacuum stirrer (e.g. Reitel Retomix model modified to run continuously)
Balance scales

### Method

i. For quality control purposes, it may assist to re-measure the solids content of the 5% w/w stock of Example 1, following the same protocol as set out in Example 1 (i.e., fully drying samples of the gel and determining the solids content, using equipment such as a Moisture balance, or drying out in a laboratory oven). This figure is recorded as a percentage (SC_{CG}).
ii. Calculate the quantity of any additional chilled (4 °C) 0.01M HCl required to generate the 1.25% by weight solids content collagen dope. This is dependent upon the ultimate volume of dope that is required, using the formula below:

SC_{CG}×M_{CG}=SC_{CD}×M_{CD}
Where:
SC_{CD}=required solids content of the collagen dope (1.25% by weight)
M_{CD}=desired mass of the collagen dope (in grams)
SC_{CG}=solids content of the collagen gel (in %)
M_{CG}=mass of the collagen gel (in grams)
   Worked Example for a desired mass of 400 g of collagen dope at 1.25% by weight solids:

(SC_{CD}×400 g)/SC_{CG}=(1.25×400)/5=100 g collagen gel (at 5% solids)

400−(SC_{CD}×M_{CD})/SC_{CG}=400−100=300 g of 0.01M HCl
iii. Add the chilled 5% w/w collagen solution and the calculated amount of chilled 0.01M HCl to a vacuum blender. Homogenise for 5 minutes. This technique may avoid the introduction of air bubbles as the two components are vigorously mixed. As an alternative, a temperature controlled industrial scale vacuum homogeniser could be used.
iv. Transfer the mixture to the degassing pot of a vacuum stirrer container and stir under 90% vacuum at 300 rpm for 60 minutes. Check for removal of all air bubbles. A higher vacuum may risk water evaporating from the mixture (thus altering the solids content of the dope). The container should be immersed in ice water to ensure the temperature remains <10 °C. Stir longer as necessary.
v. Remove and chill at 4 °C. The 1.25% w/w dope can be stored at 4 °C over an extended time period, for example a week.

### Example 3—Extrusion of Collagen Filament

Degassed 1.25% w/w collagen dope is transferred to the extrusion pressure vessel under negative pressure, of −90 kPA, to prevent air pockets forming between the viscous collagen dope and the walls of the extrusion pressure vessel (pockets of air within the collagen dope may risk discontinuous fibre extrusion). A gasket constructed of TPU (70 A Shore hardness) is used to facilitate transfer under negative pressure.

The extrusion pressure vessel is configured to withstand a pressure of up to 6 bar, being constructed from a resin that has a high tensile modulus, a high tensile strength, thermal resistance and chemical resistance (Formlabs Rigid 10K resin, constructed using a Formlabs Form 3 stereolithographic SLA printer, obtainable from Formlabs, 35 Medford St., Somerville, MA 02143, USA).

The pressure vessel tapers from the mid-section downwards to facilitate the flow of 1.25% w/w collagen dope into a 1.59 mm inner diameter tube, whilst avoiding or mitigating cavitation, thus avoiding the introduction of air bubbles into the collagen dope which could render discontinuous the resultant extrudate. The top and base supports are constructed of solid aluminium. The top contains an o-ring recess and o-ring to facilitate a pressure seal and also threaded holes to facilitate the pressured air input. The base contains a supporting block and a recess to facilitate the pipework (that contains the collagen dope and is headed towards the extrusion nozzle). The rods are M4 thread and once the top and base are in place, M4 bolts and M4 wing nut bolts are used to keep the base and top securely in place whilst the vessel is pressurised. It will be appreciated by those skilled in the art that suitable alternative materials and conditions could alternatively be used (for example, a suitable grade of stainless steel may replace the aluminium of the top support, base supports and the pressure vessel itself).

The extrusion pressure vessel (and collagen dope within it) are housed within a temperature control chamber and held at a temperature of 3-4 °C for a minimum of 12 hours prior to extrusion (unpressurised) and during extrusion itself. This may have one or more of the following advantages:
to prevent degradation, as collagen dope may start to degrade (gelatinise) if kept at higher temperatures;
to reduce the solubility of any remaining air present within the collagen dope, to mitigate or avoid the formation of discrete air bubbles;
at constant temperatures, the viscosity of the collagen dope remains constant and therefore the speed of the collagen dope through the tube remains constant at any given pressure.

Although collagen dope viscosity increases as temperature decreases, advantages of the range of from 3-4 °C, described above, outweigh the ease of extrusion observed at higher temperatures associated with lower viscosity.

The tube from the base of the extrusion pressure vessel to the extrusion nozzle is made of PTFE and has an inner diameter of 1.59 mm and an outer diameter of 3.18 mm. The use of the PTFE flexible tubing may have one or both of the following advantages:
to keep the friction level within the tube low;
to prevent an expansion of the tube at the pressures used.

Both sets of tubing (for (i) incoming pressurised air at the top of the extrusion vessel; and (ii) collagen dope extrusion at the bottom of the extrusion vessel) are connected to the extrusion vessel by flangeless fittings that include both a nut and ferrule, which are dependable, easy to use and easy to replace.

Compressed air is fed to the extrusion vessel to drive extrusion of the 1.25% w/w collagen solution. The compressed air supply is provided by a Clarke International Compressor Shhh Air 150/100 (obtainable from Clarke International, Hemnall Street, Epping, Essex, CM16 4LG, U.K.).

Pressure is controlled through a channel of an Elveflow OB1 MK3+ microfluidic multi-channel pressure & microfluidic flow controller (obtainable from Elveflow, 172 Rue de Charonne, 75011, Paris, France). Air pressure within the extrusion vessel is set to 5,450 mbar, providing an extrusion rate of the 1.25% w/w collagen solution (from the base of the extrusion pressure vessel) of 1 m/min. It will be appreciated that, should higher extrusion rates be required, the pressure may be increased.

The PTFE tube from the base of the extrusion pressure vessel is connected directly to an extrusion nozzle (also known as an extrusion needle). The extrusion nozzle has an inner diameter of 1.067 mm and a length of 38 mm. The nozzle is connected to the PTFE tube by flangeless fittings (nut and ferrule) connected to a Male Luer Lock to Female Adapter. The extrusion nozzle then screws into the Male Luer Lock to Female Adapter. The nozzle PTFE tube are together secured in a thin walled cylinder and connected to a plotter device.

The pressure is slowly increased to 5,450 bar (at a rate of circa 2,000 mbar per minute). Increasing the pressure too rapidly may risk stressing the extrusion vessel, increasing the risk of cavitation.

The plotter device is an AxiDraw model V3/A3 (obtainable from Evil Mad Scientist Laboratories, 1285 Forgewood Ave, Sunnyvale, CA 94089, USA). The plotter moves backward and forwards (left to right) at a speed synchronised with the speed of extrusion of collagen dope.

Thus, collagen dope is extruded onto a conveyor and the movement of the plotter allows the extrudate to be extruded onto the conveyor in the form of straight lines joined by hairpin bends, i.e. snaking in a continuous line across the conveyor. If the relative speed of the plotter is slower than the speed of extrusion, the line of extrudate is wavy where it ought to be straight (i.e., at points away from the intentional hairpin bends). If the relative speed of the plotter is faster than the speed of extrusion, the extrudate may break and/or be dragged along by the movement of the nozzle. The synchronised speed for the plotter at an extrusion pressure of 5,450 mbar is approximately 20 mm/second. The plotter moves 418 mm in each direction and the change of direction is programmed to facilitate a hairpin in the extrudate at 0 mm and 418 mm accordingly.

The control software used to control the plotter is Inkscape, an open source vector graphics editor for creating vector instructions for the plotter and communicating with the plotter in use (obtainable from https://inkscape.org).

The conveyor onto which collagen dope is extruded is approximately 440 mm wide and 950 mm in length. The purpose of the conveyor is to enable continuous movement of the extrudate along the length of the conveyor. As collagen dope is extruded along the width of the conveyor (back and forth, along a conveyor X axis), movement of the conveyor (along a conveyor Y axis) means that extrudate is extruded onto the surface of the conveyor, but not on top of previously extruded extrudate, when the plotter switches direction. The conveyor is submerged in a fibrillation and tanning solution, which contains components, described in further detail below, which facilitate both fibrillation and subsequent cross-linking of the collagen dope. The conveyor is driven by a Nema 23XL Stepper Motor and (5:1) Gearbox with an 8 mm diameter shaft. The motor is controlled by an Igus Dryve D1 Controller, which facilitates computer control (of the conveyor speed) via a browser.

The speed of the conveyor (moving along within the fibrillation and tanning solution) is configured such that extrudate is immersed in the fibrillation and tanning solution for about 60 minutes. This enables enough fibrillation and uptake of cross-linker to occur. Although more than about 60 minutes' immersion is unnecessary, it is not until after about 60 minutes' immersion that the extrudate is solid enough to handle gently. Without wishing to be bound by theory, it is thought that fibrillation occurs during submersion in the solution, as does soaking of the collagenous extrudate in cross-linker; cross-linking then occurs during drying, as moisture is lost and collagen molecules approach each other more closely.

The rotational speed of the motor/gearbox that drives the conveyor is set to 0.684° per second and transferred to the conveyor via a GT2 closed loop timing belt with a pitch of 2 mm and which is 50 mm in length and 9 mm wide.

The fibrillation and tanning solution is in a large, watertight container that is 1.03 m×0.70 m×0.1 m in dimension and will hold up to 70 litres of fluid. The container is flexible and is secured to and supported by a frame constructed of extruded aluminium (40 mm×40 mm).

The vessel is filled with 45 litres of aqueous, 0.025 M buffer solution at a pH of 8.0. Added to the solution is 45 g of glutaraldehyde and 45 g of caroxymethylcellulose (CMC).

To make the pH 8.0, 0.025 M sodium tetraborate/boric acid buffer, prepare 36000 mL of distilled water in a suitable container; ad 32.18 g sodium tetraborate; add 48.7 g boric acid. Add distilled water to make up the volume to 45 L; adjust the solution to final pH using HCl or NaOH.

The surface of the conveyor is coated with a high-friction grit to temporarily anchor the extrudate to the surface of the conveyor during the ca. 60-minute immersion period.

The conveyor belt is submerged in the solution to a level of 10 mm below the surface of the solution. Prior to commencing collagen extrusion, the conveyor belt is run for a period of 30 minutes in order to remove any trapped pockets of air (formed when the liquid is initially added to the container). If any trapped air pockets rise up to the surface, it creates a sufficient disturbance (within the solution) to interfere with the extrudate and subsequent collection of the filament (from the conveyor to the drier) becomes problematic.

Fibre extrusion is initiated by the following:
using Igus Drive Interface, ensure the conveyor is running at the correct speed;
using Inscape Axidraw Control select the number of “Copies to Plot” i.e. the number of cycles the plotter will complete, then apply same—the plotter will commence its operation;
using Elveflow, slowly build the pressure to 5,275 mbar. If the extrudate is wavy where straight lines are intended (i.e., at points away from the hairpin bends), reduce the pressure in increments of 25 mbar until straight. If the extrudate appears significantly shorter in length than 418 mm on the conveyor (in the X direction), increase the pressure by 25 mbar until the length is approximately 418 mm;
the collagen should then start being extruded onto the conveyor. Note, it is quite difficult to see when initially extruded because it is transparent. Over time, it becomes much more opaque due to fibrillation (and even more opaque once cross-linking has in due course occurred);
once approximately one hour has passed, the thread of collagenous extrudate will reach the end of the conveyor and can be removed (at a speed that is synchronized with both the extrusion speed and the speed of the conveyor) using 4 rollers that tension the filament as it is fed through to the drying equipment.

The thread of collagenous extrudate is lifted from the end of the conveyor and fed through and onto the take-off rollers. The rotation of each take-off roller is facilitated a 9 V stepper motor (0.52 A, max power 1.83 W). A (5:1) ratio gearbox is also used facilitate smooth, steady and accurate rotation of the rollers. The stepper motors are both powered and controlled via a control hub. Each control hub is connected (via Bluetooth) to overall motor-control software. Feedback control is accordingly possible, in order to:
ensure that the rotational speeds of the take-off rollers are the same; and
synchronize the speeds of the take-off rollers in order that the thread of collagenous extrudate is removed from the conveyor to the drying equipment at the same speed that it is being extruded onto the conveyor from the extrusion needle.

If the speed of removal is faster than the extrusion speed, there is a risk that the thread of collagenous extrudate will not spend enough time in the solution to fibrillate and pick up cross-linker. Additionally, the thread may be stretched and may break.

At the point of being removed from the conveyor, the thread remains wet. While fragile, it is possible to handle it, e.g. using tweezers. Its elongation at break is in a range of from about 25% to about 40%.

At the point of being removed from the fibrillation and tanning solution/conveyor, the filament is essentially 98.75% water—i.e. only 1.25% is collagen (as per the initial collagen dope). Whilst it is possible to increase the concentration of the collagen dope, fibrillation may then take longer and once dried, the diameter of the collagenous filament may increase. A dope concentration of 1.25% may facilitate practicable extrusion rates, extrusion pressures and acceptable dried collagenous filament diameters. The vast majority of the strength of the filament is only achieved subsequent to drying. Accordingly, handling of the filament during drying is critical.

Unlike the extrusion of synthetic polymers (where a very significant proportion of the extrudate strength is apparent immediately upon extrusion), subsequent to the extrusion of the collagen dope, time (approximately one hour) is needed to facilitate fibrillation and subsequent cross linking. That being said, in the present process, even this is surprisingly accelerated compared to, e.g., Haynl et al., Nano Lett. 2016, 16, 5917-5922 (requiring ca. 24 hours in a liquid of a fibrillation solution).

### Example 4—Drying System

The (typical) drying process for wet spun synthetic (e.g. polyester) continuous filament involves deliberately stretching the filament over large distances (e.g. hundreds of metres) and subjecting the filament to high drying temperatures (e.g. >>70 °C), whilst wrapping the fibre around large rollers. All three of these factors (i.e. stretching over a large distance, subjecting the filament to high temperatures and wrapping the drying filament around a roller) may be problematic for collagen, because:
It requires very little force to rupture the wet collagen; at a length of between 2 meters to 2.5 meters, the initial wet thread of collagenous extrudate breaks under its own weight. Stretching over a large distance is not feasible.
Collagen may degrade at temperatures above 74 °C. Accordingly, exposing the wet thread of collagenous extrudate to such temperatures is to be avoided.
The initial wet thread of collagenous extrudate is 98.75% water. During the drying process, the cross-section of the thread should remain substantially round. If the drying occurs while the thread of collagenous extrudate is in contact with a surface (even a dimensionally shaped surface or a low surface energy surface), the resulting collagenous filament may be flat, ribbon like and extremely weak. By drying the thread of collagenous extrudate unsupported, i.e. suspended mid-air, with little or no surface contact time, the cross-section of the filament may remain substantially round upon drying.

The present drying system is configured so that:
Lengths of thread travelling unsupported through drying cavities between rollers do not exceed 0.7 meters, mitigating against the filament breaking under its own weight.
Hot air facilitates removal of water from the thread, thus drying into a tanned collagenous filament. The air is evenly dispersed, consistent and does not excessively mechanically strain the thread. It is at a temperature of 50 °C to 65 °C the contact time of the wet thread with rollers is minimised.
Any strain induced during drying is kept safely below the breaking strain of the thread of collagenous extrudate until it is fully dried into a collagenous filament. As it is drying, there is an initial reduction in the level of strain that can be tolerated, i.e. a decrease in strain at rupture. Without wishing to be bound by theory, this is thought to be due to removal of water, which had acted as a secondary plasticiser. Subsequently, there is a step increase in strength. In other words, as the water is removed, the filament effectively becomes more fragile until a step change occurs and it becomes strong.
It is modular, to enable drying modules to be added, e.g. to facilitate faster movement through the drying system; or subtracted e.g. because they are unnecessary. An exemplary drying system comprises 8 modules. Each module comprises a number of components:
i. Rotating rollers that facilitate the movement of the filament through the drying system, between which are drying cavities.
ii. Heating matrices, also referred to herein as heaters, that evenly pass warm air over the thread of collagenous extrudate as it moves through the drying cavities.
iii. Motors, gearboxes, control hubs & motor speed controls to provide a feedback control system to adjust speed/tensioning of the thread.
iv. Framework to position and secure the various items referred to above.

Subsequently there may be a winder to wind the filament. The winder may itself be heated to provide a final “boost” to the drying of the thread into collagenous filament.

Four rollers are positioned on each drying module. The bottom right roller (A) rotates clockwise, the top right roller (B) rotates counter-clockwise, the top left roller (C) rotates counter-clockwise and the bottom left roller (D) rotates clockwise. Thus the overall movement of the thread of collagenous extrudate is from right to left, moving upward from roller (A) to roller (B) and downward from roller (C) to roller (D). The distance between the centre of roller (A) and roller (D) is 126 mm. The distance between the centre of roller (B) and roller (C) is 80 mm. The vertical distance between the centre of the upper and lower rollers is 186 mm (it is this vertical section where the heating matrices are located). After the thread of collagenous extrudate passes around the bottom left roller (D) it moves onwards to the bottom right roller (A) on the next drying module. On the last drying module (Module 8), after the thread of collagenous extrudate passes around the bottom right roller (D), it moves onward to the filament winder, where the dry collagenous filament is collected.

The heating matrices are designed to facilitate the movement of hot air at low speeds over the moving thread of collagenous extrudate. Hot air enters the rear of each matrix and into a central channel from where it is distributed through a series of channels that blow hot air over the thread of collagenous extrudate.

Each channel has an equal and opposite channel, in order that the air flow from each channel has an equal and opposite flow of hot air and the net air movement (normal to the direction of movement of the thread of collagenous extrudate) is close to zero. This reduces the potential for the movement of the hot air to cause lateral movement of the filament and reduces possible vibrational effects on the filament, thus reducing the risk of breakage.

Convection facilitates drying. The air velocity is such that an anemometer held at the outlet face of the matrix registers an air velocity of circa 0.60 to 1.0 m/s. The temperatures inside the matrix along the fibre path are ca. 55 °C.

Within each drying module, there are 2 heating matrices. Accordingly, the thread of collagenous extrudate is subject to air drying between the bottom right roller (A) and the top right roller (B) when the thread of collagenous extrudate is moving upwards and the top left roller (C) and the bottom left roller (D) when the thread of collagenous extrudate is moving downwards.

The hot air inputs to the 2 heating matrices in each drying module are joined in order that a single hot air generator can suitably be used for each heating module.

An air diffuser is also provided in each matrix to promote even heat and air distribution and also to facilitate a reduction of air velocity (to 0.60 to 1.0 m/s).

Each of the four rotating rollers within each drying module uses a 9V stepper (max 0.52 A, 1.83 W). A (5:1) ratio gearbox is also used to facilitate smooth, steady and accurate rotation of the rollers. Each of the four stepper motors (in one drying module) is powered and controlled via a control hub. Each control hub is then connected via Bluetooth to motor-control software, enabling feedback control.

The drying equipment of each module is secured to an aluminium frame that is constructed with 20 mm×20 mm extruded Al sections.

Once the thread of collagenous extrudate has passed through all the drying modules thus forming dry filament, it is transferred to a filament winding machine. The speed of the winding machine is synchronised with the speed of transferring the filament to it from the drying modules.

Where in the foregoing description, features or limitations are mentioned which have equivalents that are known, evident or foreseeable to those skilled in the art in the light of the present disclosure, then such equivalents are incorporated herein as if particularly set forth. Reference should be made primarily to the claims for determining the scope of the subject-matter of the present disclosure. The scope of protection sought by the present application further encompasses any such equivalents. It will also be appreciated by those skilled in the art that features or limitations of the disclosed subject-matter that are described as preferable, suitable, advantageous, convenient or the like may be optional and may not limit the scope of the independent claim(s) or the protection sought unless explicitly stated otherwise. Moreover, it is to be understood that such optional features or limitations, while of potential benefit in some implementations of the disclosed subject-matter, may be undesirable, and may therefore be absent or omitted in other implementations.

1. A method of forming a collagenous textile filament, comprising:
supporting a continuous thread of collagenous extrudate upon a moving build platform surface through the liquid of a fibrillation bath;
transferring the thread of collagenous extrudate to at least one drying module; and
moving the thread of collagenous extrudate through the or each drying module, wherein in the or each drying module the thread moves unsupported through a drying cavity from a first roller to a second roller spaced apart from the first roller, wherein the or each drying cavity is warmed by at least one heater.

2. The method of claim 1, wherein the at least one drying module is a plurality of drying modules.

3. The method of claim 1, comprising extruding the continuous thread of collagenous extrudate from an extrusion needle into the liquid of the bath and towards the surface of the build platform, before supporting the thread upon the moving build platform surface through the liquid of the bath, optionally wherein the extrusion needle moves relative to the surface of the moveable build platform while extruding the thread.

4-5. (canceled)

6. The method of claim 1, wherein the collagenous extrudate has a collagen content in a range of from about 0.5% w/w to about 3% w/w.

7. (canceled)

8. The method of claim 1, wherein the liquid of the fibrillation bath is an aqueous fibrillation and tanning solution.

9. The method of claim 1, wherein the drying module(s) are directly downstream of the fibrillation bath and/or wherein the fibrillation bath is the only bath upstream of the drying module(s).

10. The method of claim 1, wherein the drying module rollers rotate at synchronised speeds.

11. (canceled)

12. The method of claim 1, wherein the or each heater comprises a plurality of air channels encircling or part-encircling its drying cavity.

13. (canceled)

14. The method of claim 1, wherein the or each drying cavity is warmed up to a temperature in a range of from about 50 to about 70 °C by its heater.

15. Apparatus for forming a collagenous textile filament, comprising:
a fibrillation bath vessel containing a moveable build platform; and
at least one drying module downstream of the fibrillation bath vessel,
the moveable build platform having a surface configured to receive a continuous thread of collagenous extrudate and to move thereby supporting the received continuous thread of collagenous extrudate through the liquid of a fibrillation bath;
the or each drying module comprising a first roller and a second roller spaced apart from the first roller, a drying cavity between the rollers through which the continuous thread of extrudate is moveable unsupported during operation, and at least one heater configured to warm the or each drying cavity.

16. The apparatus of claim 15, wherein the at least one drying module is a plurality of drying modules.

17. The apparatus of claim 15, further comprising an extrusion needle for extruding the continuous thread of collagenous extrudate into the liquid of the fibrillation bath and towards the surface of the build platform, optionally wherein the extrusion needle is configured to move relative to the surface of the moveable build platform during extrusion of the thread.

18. (canceled)

20. The apparatus of claim 15, wherein the collagenous extrudate has a collagen content in a range of from about 0.5% w/w to about 3% w/w.

21. The apparatus of claim 15, wherein the build platform is configured for its surface to support the continuous thread of collagenous extrudate through the liquid of a fibrillation bath, for about 30 minutes up to about 2 hours.

22. The apparatus of claim 15, further comprising the liquid of the fibrillation bath.

23. (canceled)

24. The apparatus of claim 15, wherein the fibrillation bath vessel is directly upstream of the drying module(s) and/or wherein the fibrillation bath vessel is the only bath vessel upstream of the drying module(s).

25. The apparatus of claim 15, wherein the drying module rollers are configured to rotate at synchronised speeds.

26. (canceled) 27. The apparatus of claim 15, wherein the or each heater comprises a plurality of air channels encircling or part-encircling its drying cavity.

28-30. (canceled)

31. A collagenous textile filament produced by the method of claim 1.

32. The collagenous textile filament of claim 31, having a tenacity of about 0.5 or more grams per denier, measured in accordance with ASTM D2256.

33-36. (canceled)
